(19) United States
(12) Patent Application Publication
(10) Pub. No.: US 2008/0306096 A1
(43) Pub. Date: Dec. 11, 2008

(54) Quinazoline Derivatives, Process for Their Preparation and Their Use as Anti-Cancer Agents

(75) Inventors: Brian Aquila, Marlborough, MA (US); Jayachandran Ezhuthachan, Brookline, MA (US); Paul Lyne, Arlington, MA (US); Timothy Pontz, Cambridge, MA (US); Xiaolan Zheng, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/097,965

(22) PCT Filed: Dec. 19, 2006
(86) PCT No.: PCT/GB2006/004756

§ 371 Date: Jun. 18, 2008

Related U.S. Application Data
(60) Provisional application No. 60/753,313, filed on Dec. 22, 2005.

(51) Int. Cl. A61K 31/517; A61P 35/00; C07D 239/94; C07D 403/12; C07D 405/02; C07D 409/02; C07D 401/02

(52) U.S. Cl. 514/266.22; 544/293; 544/284; 514/266.4; 514/266.23; 514/266.24

(57) ABSTRACT

The invention relates to chemical compounds of the formula (I) or pharmaceutically acceptable salts thereof, which possess B Raf inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

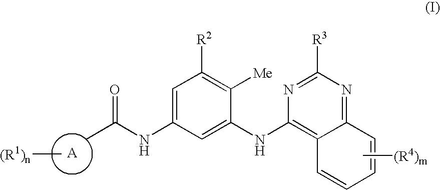

The invention relates to chemical compounds, or pharmaceutically acceptable salts thereof, which possess B-Raf inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The classical Ras, Raf, MAP protein kinase/extracellular signal-regulated kinase kinase (MEK), extracellular signal-regulated kinase (ERK) pathway plays a central role in the regulation of a variety of cellular functions dependent upon cellular context, including cellular proliferation, differentiation, survival, immortalization and angiogenesis (reviewed in Peyssonnaux and Eychene, Biology of the Cell, 2001, 93, 3-62). In this pathway, Raf family members are recruited to the plasma membrane upon binding to guanosine triphosphate (GTP) loaded Ras resulting in the phosphorylation and activation of Raf proteins. Activated Rafs then phosphorylate and activate MEKs, which in turn phosphorylate and activate ERKs. Upon activation, ERKs translocate from the cytoplasm to the nucleus resulting in the phosphorylation and regulation of activity of transcription factors such as Elk-1 and Myc.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp. Rev. Mol. Med., 2002, 25 April, http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway.

Three Raf serine/threonine protein kinase isoforms have been reported Raf-1/c-Raf, B-Raf and A-Raf (reviewed in Mercer and Pritchard, Biochim. Biophys. Acta, 2003, 1653, 25-40), the genes for which are thought to have arisen from gene duplication. All three Raf genes are expressed in most tissues with high-level expression of B-Raf in neuronal tissue and A-Raf in urogenital tissue. The highly homologous Raf family members have overlapping but distinct biochemical activities and biological functions (Hagemann and Rapp, Expt. Cell Res. 1999, 253, 34-46). Expression of all three Raf genes is required for normal murine development however both c-Raf and B-Raf are required to complete gestation. B-Raf −/− mice die at E12.5 due to vascular haemorrhaging caused by increased apoptosis of endothelial cells (Wojnowski et al., Nature Genet., 1997, 16, 293-297). B-Raf is reportedly the major isoform involved in cell proliferation and the primary target of oncogenic Ras. Activating somatic missense mutations have been identified exclusively for B-Raf, occurring with a frequency of 66% in malignant cutaneous melanomas (Davies et al., Nature, 2002, 417, 949-954) and also present in a wide range of human cancers, including but not limited to papillary thyroid tumours (Cohen et al., J. Natl. Cancer Inst., 2003, 95, 625-627), cholangiocarcinomas (Tannapfel et al., Gut, 2003, 52, 706-712), colon and ovarian cancers (Davies et al., Nature, 2002, 417, 949-954). The most frequent mutation in B-Raf (80%) is a glutamic acid for valine substitution at position 600. These mutations increase the basal kinase activity of B-Raf and are thought to uncouple Raf/MEK/ERK signalling from upstream proliferation drives including Ras and growth factor receptor activation resulting in constitutive activation of ERK. Mutated B-Raf proteins are transforming in NIH3T3 cells (Davies et al., Nature, 2002, 417, 949-954) and melanocytes (Wellbrock et al., Cancer Res., 2004, 64, 2338-2342) and have also been shown to be essential for melanoma cell viability and transformation (Hingorani et al., Cancer Res., 2003, 63, 5198-5202). As a key driver of the Raf/MEK/ERK signalling cascade, B-Raf represents a likely point of intervention in tumours dependent on this pathway.

AstraZeneca application WO 00/20402 discloses certain amide derivatives which are inhibitors of the production of cytokines such as TNF, in particular of TNFα, and various interleukins, in particular IL-1. The present inventors have surprisingly found that certain other, novel, amide derivatives are potent B-Raf inhibitors and are accordingly expected to be useful in the treatment of neoplastic disease.

Accordingly, the present invention provides a compound of formula (I):

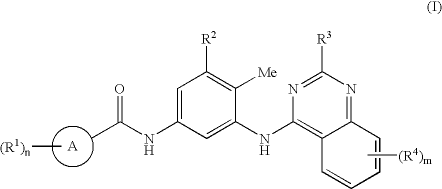

wherein:

Ring A is phenyl or a 5- or 6-membered heteroaryl; wherein if said heteroaryl contains an —NH— moiety that nitrogen may be optionally substituted by a group selected from R⁵;

R¹ is a substituent on carbon and is selected from halo, nitro, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N- (C₁₋₆alkyl)amino, C₁₋₆alkanoylamino, N-(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, N-(C₁₋₆alkyl)sulphamoyl, N,N—(C₁₋₆alkyl)₂sulphamoyl, C₁₋₆alkylsulphonylamino, carbocyclyl or carbon linked heterocyclyl; wherein R¹ may be optionally substituted on carbon by one or more R⁸; and wherein if said heterocyclyl contains an —NH— moiety that nitrogen may be optionally substituted by a group selected from R⁹;

n is selected from 1-4; wherein the values of R¹ may be the same or different;

R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁰— or heterocyclyl-R¹¹; wherein R² may be optionally substituted on carbon by one or more R¹²; and wherein if said heterocyclyl contains an —NH— moiety that nitrogen may be optionally substituted by a group selected from R¹³;

R³ and R⁴ are substituents on carbon and are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁴— or heterocyclyl-R¹⁵—; wherein R⁴ may be optionally substituted on carbon by one or more R¹⁶; and wherein if said heterocyclyl contains an —NH— moiety that nitrogen may be optionally substituted by a group selected from R¹⁷;

m is selected from 0-4; wherein the values of R⁴ may be the same or different;

R⁸ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N—(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁸— or heterocyclyl-R¹⁹—; wherein R⁸ and R¹² independently of each other may be optionally substituted on carbon by one or more R²⁰; and wherein if said heterocyclyl contains an —NH— moiety that nitrogen may be optionally substituted by a group selected from R²¹;

R¹⁶ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R¹⁰, R¹¹, R¹⁴, R¹⁵, R¹⁸, R¹⁹, R²² and R²³ are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ are independently selected from hydrogen or C₁₋₆alkyl and s is 0-2;

R⁵, R⁹, R¹³, R¹⁷, R²¹ and R²⁵ are independently selected from C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;

R²⁰ and R²⁴ are independently selected from halo, nitro, cyano, hydroxy, trifluoromethoxy, trifluoromethyl, amino, carboxy, carbamoyl, mercapto, sulphamoyl, methyl, ethyl, hydroxymethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that said compound is not N-{3-[(6,7-dimethoxyquinazolin-4-yl)amino]-4-methylphenyl}-3-(trifluoromethyl)benzamide.

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

Ring A is a “5- or 6-membered heteroaryl”. A “5- or 6-membered heteroaryl” is a fully unsaturated aromatic ring containing 5 or 6 atoms of which at least one atom is chosen from nitrogen, sulphur or oxygen. Suitably values for a “5- or 6-membered heteroaryl” include pyridyl, pyrimidyl, pyrazinyl, pyridazinyl, thienyl, furyl, pyrrolyl and imidazolyl.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxides. A carbon linked heterocyclyl is a heterocyclyl linked to the next group via a carbon atom in the heterocyclyl ring. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “CC₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N-(C₁₋₆alkyl)amino” include methylamino and ethylamino. Examples of “N,N-(C₁₋₆alkyl)₂-amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N-(C₁₋₆alkyl)sulphamoyl” are N-(methyl)sulphamoyl and N-(ethyl)sulphamoyl. Examples of “N-(C₁₋₆alkyl)₂sulphamoyl” are N,N-(dimethyl)sulphamoyl and N-(methyl)-N-(ethyl)sulphamoyl. Examples of “N-(C₁₋₆alkyl)carbamoyl” are N-(C₁₋₄alkyl)carbamoyl, methylaminocarbonyl and ethylaminocarbonyl. Examples of “N,N-(C₁₋₆alkyl)₂-carbamoyl” are N,N—(C₁₋₄alkyl)₂-carbamoyl, dimethylaminocarbonyl and methylethylaminocarbonyl. Examples of “C₁₋₆alkylsulphonyl” are mesyl, ethylsulphonyl and isopropylsulphonyl. Examples of “C₁₋₆alkylsulphonylamino” are mesylamino, ethylsulphonylamino and isopropylsulphonylamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess B-Raf inhibitory activity. The invention further relates to any and all tautomeric forms of the compounds of the formula (I) that possess B-Raf inhibitory activity.

It is also to be understood that certain compounds of the formula (I) can exist in solvated as well as unsolvated forms such as, for example, hydrated forms. It is to be understood that the invention encompasses all such solvated forms which possess B-Raf inhibitory activity.

Particular values of variable groups are as follows. Such values may be used where appropriate with any of the definitions, claims or embodiments defined hereinbefore or hereinafter.

Ring A is phenyl or a 5- or 6-membered heteroaryl.

Ring A is phenyl or a 5- or 6-membered heteroaryl; wherein if said heteroaryl contains an —NH— moiety that nitrogen may be optionally substituted by a group selected from R⁵; wherein R⁵ is C₁₋₆alkyl.

Ring A is phenyl, thienyl or pyridyl.

Ring A is phenyl, pyrazolyl, thienyl or pyridyl; wherein said pyridyl may be optionally substituted on nitrogen by a group selected from R⁵; wherein R⁵ is C₁₋₆alkyl.

Ring A is phenyl, thien-2-yl or pyrid-4-yl.

Ring A is phenyl, thien-2-yl, 1-t-butyl-1H-pyrazol-4-yl, 1-t-butyl-1H-pyrazol-5-yl or pyrid-4-yl.

R¹ is a substituent on carbon and is selected from halo, methyl, C₁₋₆alkylS(O)ₐ wherein a is 2, N,N-(C₁₋₆alkyl)₂sulphamoyl, carbocyclyl or carbon linked heterocyclyl; wherein R¹ may be optionally substituted on carbon by one or more R⁸; wherein

R⁸ is selected from halo, cyano, N,N-(C₁₋₆alkyl)₂-amino.

R¹ is a substituent on carbon and is selected from halo, C₁₋₆alkyl, C₁₋₆alkylS(O)ₐ wherein a is 2, N,N-(C₁₋₆alkyl)₂sulphamoyl, carbocyclyl or carbon linked heterocyclyl; wherein R¹ may be optionally substituted on carbon by one or more R⁸; wherein

R⁸ is selected from halo, cyano or N,N-(C₁₋₆alkyl)₂-amino.

R¹ is a substituent on carbon and is selected from fluoro, chloro, isopropyl, mesyl, N,N-dimethylsulphamoyl, cyclopropyl, cyclobutyl or carbon linked 2,3,5,6-tetrahydropyran; wherein R¹ may be optionally substituted on carbon by one or more R⁸; wherein

R⁸ is selected from fluoro, cyano, N,N-dimethylamino. R¹ is a substituent on carbon and is selected from fluoro, chloro, methyl, isopropyl, mesyl, N,N-dimethylsulphamoyl, cyclopropyl, cyclobutyl or carbon linked 2,3,5,6-tetrahydropyran; wherein R¹ may be optionally substituted on carbon by one or more R⁸; wherein

R⁸ is selected from fluoro, cyano or N,N-dimethylamino.

R¹ is a substituent on carbon and is selected from fluoro, chloro, trifluoromethyl, 1-methyl-1-cyanoethyl, 1-cyanocyclobutyl, 4-cyano-2,3,5,6-tetrahydropyran-4-yl, 1-cyanocyclopropyl, isopropyl, mesyl, N,N-dimethylsulphamoyl, dimethylaminomethyl and cyclopropyl.

R¹ is a substituent on carbon and is selected from fluoro, chloro, methyl, trifluoromethyl, 1-methyl-1-cyanoethyl, 1-cyanocyclobutyl, 4-cyano-2,3,5,6-tetrahydropyran-4-yl, 1-cyanocyclopropyl, isopropyl, mesyl, N,N-dimethylsulphamoyl, dimethylaminomethyl and cyclopropyl.

R¹ is a substituent on carbon and is selected from 1-methyl-1-cyanoethyl.

R¹ is not trifluoromethyl.

n is selected from 1 or 2; wherein the values of R¹ may be the same or different.

n is 1.

n is 2; wherein the values of R¹ may be the same or different.

R² is hydrogen.

R³ and R⁴ are substituents on carbon and are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁴— or heterocyclyl-R¹⁵—; wherein R⁴ may be optionally substituted on carbon by one or more R¹⁶; and wherein if said heterocyclyl contains an —NH— moiety that nitrogen may be optionally substituted by a group selected from R¹⁷.

R³ and R⁴ are substituents on carbon and are independently selected from halo, nitro, hydroxy, amino, carboxy, C₁₋₆alkyl and C₁₋₆alkoxy; wherein R⁴ may be optionally substituted on carbon by one or more R¹⁶;

R¹⁶ is selected from halo, amino, N,N—(C₁₋₆alkyl)₂-amino, C₁₋₆alkoxycarbonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R²² and R²³ are independently selected from a direct bond and —O—;

R²⁵ is selected from C₁₋₆alkyl and C₁₋₆alkoxycarbonyl;

R²⁴ is hydroxymethyl.

R³ and R⁴ are substituents on carbon and are independently selected from hydrogen, halo, nitro, hydroxy, amino, carboxy, C₁₋₆alkyl and C₁₋₆alkoxy; wherein R⁴ may be optionally substituted on carbon by one or more R¹⁶;

R¹⁶ is selected from halo, amino, C₁₋₆alkoxy, N,N-(C₁₋₆alkyl)₂-amino, C₁₋₆alkoxycarbonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R²² and R²³ are independently selected from a direct bond and —O—;

R²⁵ is selected from C₁₋₆alkyl and C₁₋₆alkoxycarbonyl;

R²⁴ is hydroxymethyl.

R³ and R⁴ are substituents on carbon and are independently selected from fluoro, nitro, hydroxy, amino, carboxy, methyl, methoxy, ethoxy, propoxy and isopropoxy; wherein R⁴ may be optionally substituted on carbon by one or more R¹⁶;

R¹⁶ is selected from fluoro, bromo, amino, N,N-dimethylamino, t-butoxyoxycarbonylamino, phenyl-R²²—, piperidinyl-R²³—, azetidinyl-R²³—, pyrrolidinyl-R²³— or morpholino-R²³—; wherein R¹⁶ may be optionally substituted on carbon by one or more R²⁴; and wherein said pyrrolidinyl or piperidinyl may be optionally substituted on nitrogen by a group selected from R²⁵;

R²² and R²³ are independently selected from a direct bond and —O—;

R²⁵ is selected from methyl and t-butoxycarbonyl;

R²⁴ is hydroxymethyl.

R³ and R⁴ are substituents on carbon and are independently selected from hydrogen, fluoro, chloro, bromo, nitro, hydroxy, amino, carboxy, methyl, methoxy, ethoxy, propoxy and isopropoxy; wherein R⁴ may be optionally substituted on carbon by one or more R¹⁶;

R¹⁶ is selected from fluoro, bromo, amino, methoxy, N,N-dimethylamino, t-butoxyoxycarbonylamino, phenyl-R²²—, piperidinyl-R²³—, azetidinyl R²³—, pyrrolidinyl-R²³— or morpholino-R²³—; wherein R¹⁶ may be optionally substituted on carbon by one or more R²⁴; and wherein said pyrrolidinyl, azetidinyl or piperidinyl may be optionally substituted on nitrogen by a group selected from R²⁵;

R²² and R²³ are independently selected from a direct bond and —O—;

R²⁵ is selected from methyl and t-butoxycarbonyl;

R²⁴ is hydroxymethyl.

R³ and R⁴ are substituents on carbon and are independently selected from fluoro, nitro, hydroxy, amino, carboxy, methoxy, benzyloxy, 3-aminopropoxy, 3-morpholinopropoxy, 1-methylpyrrolidin-2-ylmethoxy, piperidin-4-ylmethoxy, piperidin-3-ylmethoxy, azetidin-2-ylmethoxy, azetidin-3-ylmethoxy, pyrrolidin-2-ylmethoxy, pyrrolidin-3-yloxy, 2-(2-hydroxymethylpyrrolidin-1-yl)ethoxy, 3-(2-hydroxymethylpyrrolidin-1-yl)propoxy, 3-dimethylaminopropoxy, trifluoromethyl, propoxy, isopropoxy, 3-(t-butoxycarbonylamino)propoxy, 3-bromopropoxy, 1-(t-butoxycarbonyl)piperidin-4-ylmethoxy and 1-(t-butoxycarbonyl)piperidin-3-ylmethoxy.

R³ and R⁴ are substituents on carbon and are independently selected from hydrogen, fluoro, chloro, bromo, nitro, hydroxy, amino, carboxy, methyl, methoxy, benzyloxy, 3-aminopropoxy, 3-morpholinopropoxy, 2-methoxyethoxy, 1-methylpyrrolidin-2-ylmethoxy, piperidin-4-ylmethoxy, piperidin-3-ylmethoxy, azetidin-2-ylmethoxy, 1-t-butoxycarbonylazetidin-2-ylmethoxy, azetidin-3-ylmethoxy, 1-t-butoxycarbonylazetidin-3-ylmethoxy, pyrrolidin-2-ylmethoxy, 1-t-butoxycarbonylpyrrolidin-2-ylmethoxy, pyrrolidin-3-yloxy, 1-t-butoxycarbonylpyrrolidin-3-yloxy, 2-(2-hydroxymethylpyrrolidin-1-yl)ethoxy, 3-(2-hydroxymethylpyrrolidin-1-yl)propoxy, 3-dimethylaminopropoxy, trifluoromethyl, propoxy, isopropoxy, 3-(t-butoxycarbonylamino)propoxy, 3-bromopropoxy, 1-(t-butoxycarbonyl)piperidin-4-ylmethoxy and 1-(t-butoxycarbonyl)piperidin-3-ylmethoxy.

R³ is hydrogen.

m is selected from 0-2; wherein the values of R⁴ may be the same or different.

m is 0.

m is 1.

m is 2; wherein the values of R⁴ may be the same or different.

Therefore in a further aspect of the invention there is provided a compound of formula (I) (as depicted above) wherein:

Ring A is phenyl or a 5- or 6-membered heteroaryl;

R¹ is a substituent on carbon and is selected from halo, methyl, C₁₋₆alkylS(O)ₐ wherein a is 2, N,N-(C₁₋₆alkyl)₂sulphamoyl, carbocyclyl or carbon linked heterocyclyl; wherein R¹ may be optionally substituted on carbon by one or more R⁸;

n is selected from 1 or 2; wherein the values of R¹ may be the same or different;

R² is hydrogen;

R³ and R⁴ are substituents on carbon and are independently selected from halo, nitro, hydroxy, amino, carboxy, C₁₋₆alkyl and C₁₋₆alkoxy; wherein R⁴ may be optionally substituted on carbon by one or more R¹⁶;

m is selected from 0-2; wherein the values of R⁴ may be the same or different;

R⁸ is selected from halo, cyano, N,N-(C₁₋₆alkyl)₂-amino;

R¹⁶ is selected from halo, amino, N,N-(C₁₋₆alkyl)₂-amino, C₁₋₆alkoxycarbonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R²² and R²³ are independently selected from a direct bond and —O—;

R²⁴ is hydroxymethyl; and

R²⁵ is selected from C₁₋₆alkyl and C₁₋₆alkoxycarbonyl;

or a pharmaceutically acceptable salt thereof;
with the proviso that said compound is not N-{3-[(6,7-dimethoxyquinazolin-4-yl)amino]-4-methylphenyl}-3-(trifluoromethyl)benzamide.

Therefore in a further aspect of the invention there is provided a compound of formula (I) (as depicted above) wherein:

Ring A is phenyl or a 5- or 6-membered heteroaryl; wherein if said heteroaryl contains an —NH— moiety that nitrogen may be optionally substituted by a group selected from R⁵;

R¹ is a substituent on carbon and is selected from halo, C₁₋₆alkyl, C₁₋₆alkylS(O)ₐ wherein a is 2, N,N-(C₁₋₆alkyl)₂sulphamoyl, carbocyclyl or carbon linked heterocyclyl; wherein R¹ may be optionally substituted on carbon by one or more R⁸;

n is selected from 1 or 2; wherein the values of R¹ may be the same or different;

R² is hydrogen;

R³ and R⁴ are substituents on carbon and are independently selected from hydrogen, halo, nitro, hydroxy, amino, carboxy, C₁₋₆alkyl and C₁₋₆alkoxy; wherein R⁴ may be optionally substituted on carbon by one or more R¹⁶;

m is selected from 0-2; wherein the values of R⁴ may be the same or different;

R⁵ is C₁₋₆alkyl;

R⁸ is selected from halo, cyano or N,N-(C₁₋₆alkyl)₂-amino;

R¹⁶ is selected from halo, amino, C₁₋₆alkoxy, N,N-(C₁₋₆alkyl)₂-amino, C₁₋₆alkoxycarbonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;

R²² and R²³ are independently selected from a direct bond and —O—;

R²⁵ is selected from C₁₋₆alkyl and C₁₋₆alkoxycarbonyl;

R²⁴ is hydroxymethyl;

or a pharmaceutically acceptable salt thereof;
with the proviso that said compound is not N-{3-[(6,7-dimethoxyquinazolin-4-yl)amino]-4-methylphenyl}-3-(trifluoromethyl)benzamide.

Therefore in a further aspect of the invention there is provided a compound of formula (I) (as depicted above) wherein:

Ring A is phenyl, thien-2-yl or pyrid-4-yl;

R¹ is a substituent on carbon and is selected from fluoro, chloro, trifluoromethyl, 1-methyl-1-cyanoethyl, 1-cyanocyclobutyl, 4-cyano-2,3,5,6-tetrahydropyran-4-yl, 1-cyanocyclopropyl, isopropyl, mesyl, N,N-dimethylsulphamoyl, dimethylaminomethyl and cyclopropyl;

n is selected from 1 or 2; wherein the values of R¹ may be the same or different;

R² is hydrogen;

R³ and R⁴ are substituents on carbon and are independently selected from fluoro, nitro, hydroxy, amino, carboxy, methoxy, benzyloxy, 3-aminopropoxy, 3-morpholinopropoxy, 1-methylpyrrolidin-2-ylmethoxy, piperidin-4-ylmethoxy, piperidin-3-ylmethoxy, azetidin-2-ylmethoxy, azetidin-3-ylmethoxy, pyrrolidin-2-ylmethoxy, pyrrolidin-3-yloxy, 2-(2-hydroxymethylpyrrolidin-1-yl)ethoxy, 3-(2-hydroxymethylpyrrolidin-1-yl)propoxy, 3-dimethylaminopropoxy, trifluoromethyl, propoxy, isopropoxy, 3-(t-butoxycarbonylamino)propoxy, 3-bromopropoxy, 1-(t-butoxycarbonyl)piperidin-4-ylmethoxy and 1-(t-butoxycarbonyl)piperidin-3-ylmethoxy;

m is selected from 0-2; wherein the values of R⁴ may be the same or different;

or a pharmaceutically acceptable salt thereof;
with the proviso that said compound is not N-{3-[(6,7-dimethoxyquinazolin-4-yl)amino]-4-methylphenyl}-3-(trifluoromethyl)benzamide.

Therefore in a further aspect of the invention there is provided a compound of formula (I) (as depicted above) wherein:

Ring A is phenyl, thien-2-yl, 1-t-butyl-1H-pyrazol-4-yl, 1-t-butyl-1H-pyrazol-5-yl or pyrid-4-yl;

R¹ is a substituent on carbon and is selected from fluoro, chloro, methyl, trifluoromethyl, 1-methyl-1-cyanoethyl, 1-cyanocyclobutyl, 4-cyano-2,3,5,6-tetrahydropyran-4-yl, 1-cyanocyclopropyl, isopropyl, mesyl, N,N-dimethylsulphamoyl, dimethylaminomethyl and cyclopropyl;

n is selected from 1 or 2; wherein the values of R¹ may be the same or different;

R² is hydrogen;

R³ and R⁴ are substituents on carbon and are independently selected from hydrogen, fluoro, chloro, bromo, nitro, hydroxy, amino, carboxy, methyl, methoxy, benzyloxy, 3-aminopropoxy, 3-morpholinopropoxy, 2-methoxyethoxy, 1-methylpyrrolidin-2-ylmethoxy, piperidin-4-ylmethoxy, piperidin-3-ylmethoxy, azetidin-2-ylmethoxy, 1-t-butoxycarbonylazetidin-2-ylmethoxy, azetidin-3-ylmethoxy, 1-t-butoxycarbonylazetidin-3-ylmethoxy, pyrrolidin-2-ylmethoxy, 1-t-butoxycarbonylpyrrolidin-2-ylmethoxy, pyrrolidin-3-yloxy, 1-t-butoxycarbonylpyrrolidin-3-yloxy, 2-(2-hydroxymethylpyrrolidin-1-yl)ethoxy, 3-(2-hydroxymethylpyrrolidin-1-yl)propoxy, 3-dimethylaminopropoxy, trifluoromethyl, propoxy, isopropoxy, 3-(t-butoxycarbonylamino)propoxy, 3-bromopropoxy, 1-(t-butoxycarbonyl)piperidin-4-ylmethoxy and 1-(t-butoxycarbonyl)piperidin-3-ylmethoxy;

m is selected from 0-2; wherein the values of R⁴ may be the same or different;

or a pharmaceutically acceptable salt thereof;
with the proviso that said compound is not N-{3-[(6,7-dimethoxyquinazolin-4-yl)amino]-4-methylphenyl}-3-(trifluoromethyl)benzamide.

In another aspect of the invention, preferred compounds of the invention are any one of the Examples or a pharmaceutically acceptable salt thereof.

Another aspect of the present invention provides a process for preparing a compound of formula (I) or a pharmaceutically acceptable salt thereof which process (wherein variable are, unless otherwise specified, as defined in formula (I)) comprises of: Process a) reacting an amine of the formula (II)

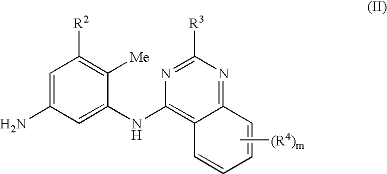

with an acid of formula (III):

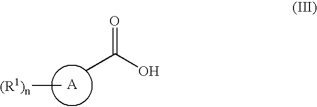

or an activated acid derivative thereof;
Process b) reacting an amine of formula (IV):

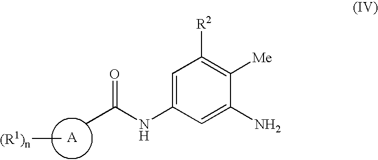

with a compound of formula (V):

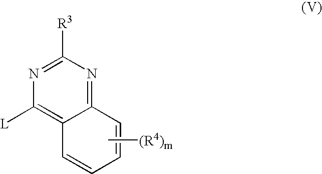

wherein L is a displaceable group
Process c) reacting an amine of formula (VI):

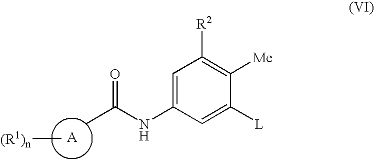

with a compound of formula (VII);

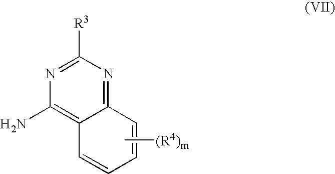

and thereafter if necessary:
i) converting a compound of the formula (I) into another compound of the formula (I);
ii) removing any protecting groups;
iii) forming a pharmaceutically acceptable salt.

L is a displaceable group, suitable values for L are for example, a halo, for example a chloro, bromo or iodo.

G is a displaceable group, suitable values for G are for example, a halo, for example a chloro, bromo or iodo; tosyl or mesyl.

Specific reaction conditions for the above reactions are as follows.

Process a) Amines of formula (II) and acids of formula (III) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 50° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 50° C.

Amines of formula (II) may be prepared according to Scheme 1:

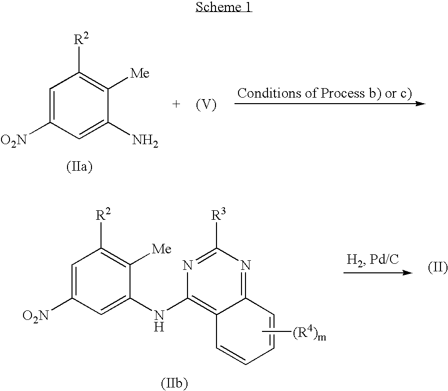

Compounds of formula (IIa) and (III) are commercially available compounds, or they are known in the literature or they may be prepared by standard processes known in the art. Process b) and Process c) Compounds of formula (IV) and (V) and compounds of formula (VI) and (VII) can be reacted together by coupling chemistry utilizing an appropriate catalyst and ligand such as Pd₂(dba)₃ and BINAP respectively and a suitable base such as sodium tert-butoxide. The reaction usually requires thermal conditions often in the range of 80° C. to 100° C.

Compounds of formula (VI) may be prepared according to Scheme 2:

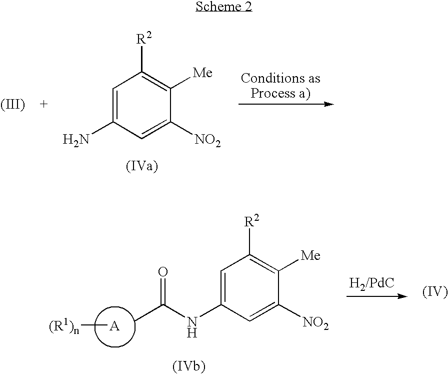

Compounds of formula (VI) may be prepared by a modification of Scheme 2).

Compounds of formula (V), (VII) and (IVa) are commercially available compounds, or they are known in the literature or they may be prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifing group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess anti-cancer activity which is believed to arise from the B-Raf inhibitory activity of the compounds. These properties may be assessed, for example, using the procedure set out below.

### B-Raf In Vitro ELISA Assay

Activity of human recombinant, purified wild type His-B-Raf protein kinase was determined in vitro using an enzyme-linked immunosorbent assay (ELISA) assay format, which measures phosphorylation of the B-Raf substrate, human recombinant, purified His-derived (detagged) MEK1. The reaction utilized 2.5 nM B-Raf, 0.15 μM MEK1 and 10 μM adenosine triphosphate (ATP) in 40 mM N-(2-hydroxyethyl)piperazine-N′-(2-ethanesulfonic acid hemisodium salt (HEPES), 5 mM 1,4-dithio-DL-threitol (DTT), 10 mM MgCl₂, 1 mM ethylenediaminetetraacetic acid (EDTA) and 0.2 M NaCl (1×HEPES buffer), with or without compound at various concentrations, in a total reaction volume of 25 μl in 384 well plates. B-Raf and compound were preincubated in 1×HEPES buffer for 1 hour at 25° C. Reactions were initiated with addition of MEK1 and ATP in 1×HEPES buffer and incubated at 25° C. for 50 minutes and reactions stopped by addition of 10 μl 175 mM EDTA (final concentration 50 mM) in 1×HEPES buffer. 5 μl of the assay mix was then diluted 1:20 into 50 mM EDTA in 1×HEPES buffer, transferred to 384 well black high protein binding plates and incubated overnight at 4° C. Plates were washed in tris buffered saline containing 0.1% 5 Tween20 (TBST), blocked with 50 μl Superblock (Pierce) for 1 hour at 25° C., washed in TBST, incubated with 50 μl rabbit polyclonal anti-phospho-MEK antibody (Cell Signaling) diluted 1:1000 in TBS for 2 hours at 25° C., washed with TBST, incubated with 50 μl goat anti-rabbit horseradish peroxidase-linked antibody (Cell Signaling) diluted 1:2000 in TBS for 1 hour at 25° C. and washed with TBST. 50 μl of fluorogenic peroxidase substrate (Quantablu 10-Pierce) was added and following incubation for 45-60 minutes, 50 μl QuantabluSTOP (Pierce) was added. Blue fluorescent product was detected at excitation 325 and emission 420 using a TECAN Ultra plate reader. Data was graphed and IC₅₀s calculated using Excel Fit (Microsoft).

When tested in the above in vitro assay, the compounds of the present invention exhibited activity less than 30 μM. For example the following results were obtained:

| Example No | IC₅₀ (μM) |
|---|---|
| Example 5 | 1,100 nM |
| Example 7 | 193 nM |
| Example 15 | 370 nM |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their B-Raf inhibitory properties. Accordingly the compounds of the present invention are expected to be useful in the treatment of diseases or medical conditions mediated alone or in part by B-Raf, i.e. the compounds may be used to produce a B-Raf inhibitory effect in a warm-blooded animal in need of such treatment.

Thus the compounds of the present invention provide a method for treating cancer characterised by inhibition of B-Raf, i.e. the compounds may be used to produce an anti-cancer effect mediated alone or in part by the inhibition of B-Raf.

Such a compound of the invention is expected to possess a wide range of anti-cancer properties as activating mutations in B-Raf have been observed in many human cancers, including but not limited to, melanoma, papillary thyroid tumors, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those primary and recurrent solid tumours which are associated with B-Raf, especially those tumours which are significantly dependent on B-Raf for their growth and spread, including for example, certain tumours of the skin, colon, thyroid, lungs and ovaries. Particularly the compounds of the present invention are useful in the treatment of melanomas.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a B-Raf inhibitory effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I), or a pharmaceutically acceptable salt thereof, as defined hereinbefore in the production of a B-Raf inhibitory effect in a warm-blooded animal such as man.

According to this aspect of the invention there is provided the use of a compound of the formula (I), or a pharmaceutically acceptable salt thereof, as defined hereinbefore in the production of an anti-cancer effect in a warm-blooded animal such as man.

According to a further feature of the invention, there is provided the use of a compound of the formula (I), or a pharmaceutically acceptable salt thereof, as defined herein before in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

The B-Raf inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegaflir, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αᵥβ₃ function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies;
(x) Cell cycle inhibitors including for example CDK inhibitiors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of B-Raf in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are the millimolar ratio equivalents to those used in the previous example;
(xi) the following abbreviations have been used:
HATU O-(7-Azabenzotriazol-1-yl)-N,N,N′,N′-tetramethyluronium hexafluorophosphate;
THF tetrahydrofuran;
DMF N,N-dimethylformamide;
EtOAc ethyl acetate;
DIEA N,N-diisopropylethylamine;
DCM dichloromethane;
DMSO dimethylsulphoxide;
MeCN acetonitrile;
TFA trifluoroacetic acid;
DIAD diisopropyl azodicarboxylate;
MeOH methanol;
   (xii) “ISCO” refers to normal phase flash column chromatography using 12 g and 40 g pre-packed silica gel cartridges used according to the manufacturers instruction obtained from ISCO, Inc, 4700 superior street Lincoln, Nebr., USA.; and
   (xiii) “Gilson HPLC” refers to a YMC-AQC18 reverse phase HPLC Column with dimension 20 nm/100 and 50 mm/250 in water/MeCN with 0.1% TFA as mobile phase, obtained
   (xiv) Parr Hydrogenator or Parr shaker type hydrogenators are systems for treating chemicals with hydrogen in the presence of a catalyst at pressures up to 5 atmospheres (60 psig) and temperatures to 80° C.

### Example 1

### 4-[(2-Methyl-5-{[3-(trifluoromethyl)benzoyl]amino}phenyl)amino]quinazoline-7-carboxylic acid

4-[(5-Amino-2-methylphenyl)amino]quinazoline-7-carboxylic acid (Method 7; 0.100 g, 0.340 mmol), 3-(trifluoromethyl)benzoyl chloride (0.062 ml, 0.408 mmol, 1.2 equiv) and disopropylethylamine (0.071 ml, 0.510 mmol, 1.5 equiv) were combined in DCM (2 ml) and stirred for 4 h at 25° C. The reaction mixture was concentrated under reduced pressure and purified by reverse phase preparative HPLC (0.1% TFA in MeCN and water). NMR: 10.58 (s, 1H), 8.75 (s, 1H), 8.68 (d, 1H), 8.39 (s, 1H), 8.26 (m, 3H), 8.19 (d, 1H), 7.97 (d, 1H), 7.90 (d, 1H), 7.79 (m, 2H), 7.64 (d, 1H), 7.38 (d, 1H), 2.18 (s, 3H); m/z 467.

### Examples 2-3

The following compound was prepared by the procedure of Example 1 utilizing the appropriate SMs.

| Ex. | Compound | ¹H NMR | m/z | SM |
|---|---|---|---|---|
| 2 | 4-({5-[(3,4-Dichloro | 10.45 (s, 1H), 8.75 (s, 1H), | 468 | 3,4-Dichloro |
| | benzoyl)amino]-2- | 8.65 (d, 1H), 8.37 (s, 1H), | | benzoic acid and |
| | methylphenyl}amino) | 8.19 (m, 3H), 7.80 (m, 3H), 7.56 (d, | | Method 7 |
| | quinazoline-7- | 1H), 7.32 (d, 1H), 2.15 (s, 3H) | | |
| | carboxylic acid | | | |
| 3 | 1-tert-Butyl-N-{3-[(6,7- | 1.05 (s, 1H), 9.65 (s, 1H), | 475 | Method 8 and 1- |
| | dimethoxyquinazolin-4- | 8.68 (s, 1H), 8.02 (s, 1H), 7.85 (d, | | tert-butyl-3- |
| | yl)amino]-4- | 1H), 7.62 (dd, 1H), 7.29 (d, | | methyl-1H- |
| | methylphenyl}-3- | 1H), 7.23 (s, 1H), 6.50 (s, 1H), | | pyrazole-5- |
| | methyl-1H-pyrazole-5- | 3.93 (d, 6H), 2.40 (s, 3H), | | carbonyl chloride |
| | carboxamide | 2.09 (s, 3H), 1.57 (s, 9H) | | |

### Example 4

### 3-(1-Cyano-1-methylethyl)-N-{3-[(6,7-dimethoxyquinazolin-4-yl)amino]-4-methylphenyl}benzamide

A solution of 4-chloro-6,7-dimethoxyquinazoline (50 mg, 0.170 mmol) and N-(3-amino-4-methylphenyl)-3-(1-cyano-1-methylethyl)benzamide (Method 24, 38 mg, 0.170 mmol) in EtOH (2 ml) was heated to 90° C. for 12 h. The organics were removed under reduced pressure. The resulting solid was purified by reverse phase preparative HPLC (0.1% TFA in MeCN and water) to give 82 mg of solid (95%). NMR: 11.07 (s, 1H), 10.43 (s, 1H), 8.73 (s, 1H), 8.05 (m, 2H), 7.91 (m, 2H), 7.75 (d, 1H), 7.61 (m, 2H), 7.39 (d, 1H), 7.27 (s, 1H), 4.00 (s, 3H), 3.98 (s, 3H), 2.17 (s, 3H), 1.74 (s, 6H); m/z 482.

### Example 5

The following compound was prepared by the procedure of Example 4 using the appropriate SMs.

| Ex. | Compound | ¹H NMR | m/z | SM |
|---|---|---|---|---|
| 5 | N-(3-{[7- | 11.32 (s, 1H), 10.47 (s, 1H), | 558 | 7-(Benzyloxy)-4- |
| | (Benzyloxy)-6- | 8.71 (s, 1H), 8.24 (s, 1H), 8.04 (s, | | chloro-6-methoxy- |
| | methoxyquinazolin-4- | 1H), 7.93 (d, 1H), 7.89 (s, 1H), | | quinazoline and |
| | yl]amino}-4- | 7.74 (d, 1H), 7.65 (d, 1H), | | Method 24 |
| | methylphenyl)-3-(1- | 7.59 (t, 1H), 7.52 (m, 2H), 7.42 (m, | | |
| | cyano-1-methylethyl)- | 5H), 5.35 (s, 2H), 4.00 (s, 3H), | | |
| | benzamide | 2.17 (s, 3H), 1.74 (s, 6H) | | |

### Example 6

### 3-(1-Cyano-1-methylethyl)-N-{3-[(7-hydroxy-6-methoxyquinazolin-4-yl)amino]-4-methylphenyl}benzamide

A solution of N-(3-{[7-(benzyloxy)-6-methoxyquinazolin-4-yl]amino}-4-methylphenyl)-3-(1-cyano-1-methylethyl)benzamide (Example 5; 50 mg, 0.084 mmol) in MeOH (2 ml) and 30% Pd/C (20 mg) was treated with hydrogen. The mixture was allowed to stir at 25° C. for 12 h before being filtered through diatomaceous earth and concentrated under reduced pressure. The resulting solid was purified by reverse phase preparative HPLC (0.1% TFA in MeCN and water) to give 30 mg of solid (71%). NMR: 11.58 (s, 1H), 11.03 (s, 1H), 10.42 (s, 1H), 8.68 (s, 1H), 8.03 (m, 2H), 7.92 (d, 1H), 7.88 (s, 1H), 7.74 (d, 1H), 7.60 (m, 2H), 7.38 (d, 1H), 7.18 (s, 1H), 3.98 (s, 3H), 2.16 (s, 3H), 1.73 (s, 6H); m/z 468.

### Example 7

### 3-(1-Cyanocyclobutyl)-N-{3-[(6,7-dimethoxyquinazolin-4-yl)amino]-4-methylphenyl}benzamide

A solution of N³-(6,7-dimethoxyquinazolin-4-yl)-4-methylbenzene-1,3-diamine (Method 8; 99 mg, 0.318 mmol), 3-(1-cyanocyclobutyl)benzoic acid (Method 17; 64 mg, 0.318 mmol) and DIEA (166 μL, 0.954 mmol, 3.0 equiv) in DMF (2 ml) was treated with HATU (145 mg, 0.382 mmol, 1.2 equiv). The reaction stirred at 50° C. for 12 h. The reaction was quenched with H₂O and extracted with EtOAc. The organics were dried with NaCl(sat) and Na₂SO₄(s) and removed under reduced pressure. The resulting solid was purified by reverse phase preparative HPLC (0.1% TFA in MeCN and water) to give 57 mg (39%). NMR: 11.04 (s, 1H), 10.44 (s, 1H), 8.72 (s, 1H), 8.07 (s, 1H), 7.94 (m, 3H), 7.71 (m, 1H), 7.61 (m, 2H), 7.39 (d, 1H), 7.24 (s, 1H), 4.00 (s, 3H), 3.98 (s, 3H), 2.77 (m, 2H), 2.67 (m, 2H), 2.30 (m, 1H), 2.17 (s, 3H), 2.03 (m, 1H); m/z 494.

### Examples 8-19

The following compounds were prepared by the procedure of Example 7 using the appropriate SMs.

| Ex. | Compound | ¹H NMR | m/z | SM |
|---|---|---|---|---|
| 8 | 3-(4-Cyanotetrahydro- | 11.08 (s, 1H), 10.42 (s, 1H), 8.73 (s, | 524 | Method 8 |
| | 2H-pyran-4-yl)-N-{3- | 1H), 8.07 (m, 2H), 7.95 (t, 1H), 7.90 (s, | | and |
| | [(6,7-dimethoxy- | 1H), 7.80 (m, 1H), 7.61 (m, 2H), | | Method |
| | quinazolin-4-yl)amino]-4- | 7.39 (d, 1H), 7.25 (s, 1H), 4.01 (m, 8H), | | 18 |
| | methylphenyl}benzamide | 3.68 (m, 2H), 2.17 (s, 3H), 2.13 (m, 4H) | | |
| 9 | 3-(1-Cyanocyclopropyl)- | 11.09 (s, 1H), 10.41 (s, 1H), 8.73 (s, | 480 | Method 8 |
| | N-{3-[(6,7-dimethoxy- | 1H), 8.08 (m, 2H), 7.87 (m, 3H), | | and |
| | quinazolin-4-yl)amino]-4- | 7.62 (m, 1H), 7.54 (m, 2H), 7.38 (d, 1H), | | Method |
| | methylphenyl}benzamide | 7.25 (s, 1H), 4.00 (s, 3H), 3.98 (s, 3H), | | 19 |
| | | 2.16 (s, 3H), 1.81 (m, 2H), 1.60 (m, 2H) | | |
| 10 | 3-Cyclopropyl-N-{3- | 11.08 (s, 1H), 10.35 (s, 1H), 8.73 (s, | 473 | Method 8 |
| | [(6,7-dimethoxy- | 1H), 8.07 (s, 1H), 7.89 (m, 1H), | | and |
| | quinazolin-4-yl)amino]-4- | 7.61 (m, 1H), 7.48 (m, 2H), 7.38 (d, 1H), | | Method |
| | methylphenyl}-5- | 7.25 (s, 1H), 7.15 (m, 1H), 4.00 (s, 3H), | | 56 |
| | fluorobenzamide | 3.98 (s, 3H), 2.16 (s, 3H), 2.04 (m, 1H), | | |
| | | 1.03 (m, 2H), 0.79 (m, 2H) | | |
| 11 | N-{3-[(6,7-Dimethoxy- | 10.31 (s, 1H), 8.51 (s, 1H), 7.94 (s, 1H), | 457 | Method 8 |
| | quinazolin-4-yl)amino]-4- | 7.72 (m, 1H), 7.65 (m, 1H), 7.52 (m, | | and |
| | methylphenyl}-3- | 2H), 7.41 (m, 3H), 7.18 (s, 1H), 3.94 (s, | | Method |
| | isopropylbenzamide | 3H), 3.93 (s, 3H), 2.91 (m, 1H), 2.11 (s, | | 22 |
| | | 3H), 1.17 (d, 6H) | | |
| 12 | N-{3-[(6,7-Dimethoxy- | 10.52 (s, 1H), 9.42 (s, 1H), 8.28 (m, | 522 | Method 8 |
| | quinazolin-4-yl)amino]-4- | 2H), 7.94 (m, 2H), 7.82 (m, 3H), | | and |
| | methylphenyl}-3- | 7.62 (m, 1H), 7.30 (d, 1H), 7.16 (s, 1H), | | Method |
| | [(dimethylamino)- | 3.93 (s, 3H), 3.92 (s, 3H), 2.64 (s, 6H), | | 25 |
| | sulfonyl]benzamide | 2.16 (s, 3H) | | |
| 13 | N-{3-[(6,7-Dimethoxy- | 10.53 (s, 1H), 9.43 (s, 1H), 8.46 (m, | 493 | Method 8 |
| | quinazolin-4-yl)amino]-4- | 1H), 8.28 (m, 2H), 8.12 (m 1H), | | and 3- |
| | methylphenyl}-3- | 7.82 (m, 3H), 7.60 (m, 1H), 7.30 (d, 1H), | | methylsulphonylbenzoic |
| | (methylsulfonyl)- | 7.16 (s, 1H), 3.92 (s, 3H), 3.91 (s, 3H), | | acid |
| | benzamide | 3.28 (s, 3H), 2.15 (s, 3H) | | |
| 14 | 5-(1-Cyano-1- | 10.28 (s, 1H), 9.43 (s, 1H), 8.28 (s, 1H), | 489 | Method 8 |
| | methylethyl)-N-{3-[(6,7- | 7.94-7.83 (m, 3H), 7.80 (s, 1H), 7.53 (d, | | and |
| | dimethoxyquinazolin-4- | 1H), 7.30-7.16 (m, 2H), 3.92 (s, 3H), | | Method |
| | yl)amino]-4-methyl- | 3.91 (s, 3H), 2.14 (s, 3H), 1.78 (s, 6H) | | 29 |
| | phenyl}thiophene-2- | | | |
| | carboxamide | | | |
| 15 | N-{3-[(6,7-Dimethoxy- | 8.49 (s, 1H), 7.93-7.95 (m, 1H), | 500 | Method 8 |
| | quinazolin-4-yl)amino]-4- | 7.78-7.84 (m, 3H), 7.28-7.45 (m, 3H), | | and 2- |
| | methylphenyl}-2-fluoro- | 3.98 (s, 3H), 3.96 (s, 3H), 2.16 (s, 3H) | | fluoro-5- |
| | 5-(trifluoromethyl)- | | | trifluoro- |
| | benzamide | | | benzoic |
| | | | | acid. |
| 16 | N-{3-[(6,7-Dimethoxy- | 8.48 (s, 1H), 8.02 (s, 1H), 7.88 (d, 1H, | 500 | Method 8 |
| | quinazolin-4-yl)amino]-4- | J = 9.23 Hz), 7.85 (s, 1H), 7.62 (d, 1H, | | and 3- |
| | methylphenyl}-3-fluoro- | J = 6.97 Hz), 7.30 (d, 1H, J = 0.47 Hz), | | fluoro-5- |
| | 5-(trifluoromethyl)- | 7.14 (s, 1H), 3.98 (s, 3H), 3.96 (s, 3H), | | trifluoro- |
| | benzamide | 2.17 (s, 3H) | | benzoic |
| | | | | acid. |
| 17 | N-{3-[6,7- | 10.26 (s, 1H), 9.72 (s, 1H), 8.35 (s, 1H), | 475 | Method 8 |
| | Dimethoxyquinazolin-4- | 7.88 (s, 1H), 7.79 (m, 1H), 7.67 (m, | | and |
| | ylamino]-4- | 1H), 7.58 (m, 2H), 7.31 (m, 2H), | | Method |
| | methylphenyl}-3-fluoro- | 7.17 (s, 1H), 3.93 (m, 6H), 2.97 (m, 1H), | | 68 |
| | 5-isopropylbenzamide | 2.14 (s, 3H), 1.723 (d, 6H) | | |
| 18 | 3-Fluoro-N-{3-[(7- | 11.48 (s, 1H), 10.55 (s, 1H), 8.80 (s, | 471 | Method |
| | methoxyquinazolin-4-yl)- | 1H), 8.70 (d, 1H), 7.95 (s, 1H), 7.90 (s, | | 62 and 3- |
| | amino]-4-methylphenyl}- | 1H), 7.80 (d, 1H), 7.66 (m, 2H), 7.50 (d, | | fluoro-5- |
| | 5-(trifluoromethyl)- | 1H), 7.48 (d, 1H), 7.30 (m, 1H), 4.00 (s, | | (trifluoro- |
| | benzamide | 3H), 2.20 (s, 3H) | | methyl)- |
| | | | | benzoic |
| | | | | acid |
| 19 | 3-(1-Cyano-1- | 11.42 (s, 1H), 10.69 (s, 1H), 8.80 (s, | 469 | Method |
| | methylethyl)-2-fluoro-N- | 1H), 8.66 (d, 1H), 7.84 (s, 1H), | | 62 and |
| | {3-[(7-methoxy- | 7.68-7.60 (m, 2H), 7.58-7.52 (m, 2H), | | Method |
| | quinazolin-4-yl)amino]-4- | 7.39 (t, 2H), 7.27 (s, 1H), 4.00 (s, 3H), | | 61 |
| | methylphenyl}benzamide | 2.16 (s, 3H), 1.77 (s, 6H) | | |

### Example 20

### N-(3-{[6-Methoxy-7-(3-morpholin-4-ylpropoxy)quinazolin-4-yl]amino}-4-methylphenyl)-3-(trifluoromethyl)benzamide

A solution of N-{3-[(7-hydroxy-6-methoxyquinazolin-4-yl)amino]-4-methylphenyl}-3-(trifluoromethyl)benzamide (Example 70; 80 mg, 0.171 mmol), 3-morpholin-4-ylpropan-1-ol (28 μl, 0.205 mmol, 1.2 equiv) and Ph₃P (86 mg, 0.328 mmol, 1.9 equiv) in THF (2 ml) at 0° C. under Ar was treated with DIAD (65 μl, 0.328 mmol, 1.9 equiv). The reaction stirred for 12 h while warming to 25° C. The reaction was quenched with 10% HCl. and extracted with EtOAc. The water layer was treated with 10% NaOH and extracted with EtOAc. The organics were dried with NaCl(sat) and Na₂SO₄(s) and removed under reduced pressure. The resulting solid was purified by reverse phase preparative HPLC (0.1% TFA in MeCN and water) and by a supercritical fluid purification system. NMR: 10.59 (s, 1H), 8.69 (s, 1H), 8.26 (m, 2H), 8.18 (m, 2H), 7.97 (d, 1H), 7.92 (s, 1H), 7.79 (t, 1H), 7.62 (d, 1H), 7.38 (d, 1H), 7.31 (s, 1H), 4.30 (m, 2H), 4.00 (m, 5H), 3.76 (m, 2H), 3.51 (m, 2H), 3.35 (m, 2H), 3.12 (m, 2H), 2.31 (m, 2H), 2.17 (s, 3H); m/z 596.

### Example 21

### N-[3-(7-Benzyloxy-quinazolin-4-ylamino)-4-methyl-phenyl]-3-(cyano-dimethyl-methyl)-benzamide

A mixture of 7-benzyloxy-4-chloro-quinazoline (1.85 g, 6.8 mmol) and N-(3-amino-4-methyl-phenyl)-3-(cyano-dimethyl-methyl)-benzamide (Method 24; 2 g, 6.8 mmol) in 15 ml of isopropanol (15 ml) was refluxed for 4 h. The reaction mixture was cooled to 25° C., and the resulting solid was collected by filtration. The product was recrystallized from MeOH to give 2.6 g of a yellow solid. NMR: 11.45 (s, 1H), 10.45 (s, 1H), 8.80 (m, 2H), 8.10 (s, 1H), 7.96-7.35 (m, 13H), 5.40 (s, 2H), 2.20 (s, 3H), 1.75 (s, 6H); m/z 527.

### Example 22

### 3-({6-Methoxy-4-[(2-methyl-5-{[3-(trifluoromethyl)benzoyl]amino}phenyl)amino]quinazolin-7-yl}oxy)propan-1-aminium chloride

A solution of tert-butyl [3-({6-methoxy-4-[(2-methyl-5-{[3-(trifluoromethyl)benzoyl]amino}phenyl)amino]quinazolin-7-yl}oxy)propyl]carbamate (Example 38; 0.065 g, 0.104 mmol) in 4 M HCl in dioxane (2 ml) was stirred at 25° C. for 45 min. The reaction mixture was concentrated under reduced pressure to give the desired product. NMR: 11.62 (s, 1H), 10.66 (s, 1H), 8.72 (s, 1H), 8.41 (s, 1H), 8.28 (m, 2H), 8.11 (m, 2H), 7.96 (d, 1H), 7.90 (s, 1H), 7.78 (t, 1H), 7.68 (d, 1H), 7.42 (s, 1H), 7.37 (d, 1H), 4.30 (m, 2H), 4.02 (s, 3H), 3.00 (m, 2H), 2.17 (m, 5H); m/z 526.

### Example 23

### 3-(Cyano-dimethyl-methyl)-N-[3-(7-methoxy-quinazolin-4-ylamino)-4-methyl-phenyl]-benzamide

A mixture of 4-chloro-7-methoxy-quinazoline (Method 32; 2 g, 10.28 mmol) and N-(3-amino-4-methylphenyl)-3-(1-cyano-1-methylethyl)benzamide (Method 24; 2 g, 6.83 mmol) in isopropanol (15 ml) was refluxed for 12 h. The organics were removed under reduced pressure and the residue was purified by column chromatography utilizing an ISCO system (EtOAc) and then by reverse phase preparative HPLC (0.1% TFA in MeCN and water) to give a light yellow solid (2.1 g, 68%). NMR: 11.50 (s, 1H), 10.45 (s, 1H), 8.75 (m, 2H), 8.00-7.80 (m, 3H), 7.70-7.40 (m, 4H), 7.30 (m, 2H), 3.91 (s, 3H), 2.10 (s, 3H), 1.70 (s, 6H); m/z 451.

### Examples 24-36

The following compounds were prepared by the procedure of Example 23, using the appropriate substituted 2-amino benzoic acid as a starting material.

| Ex | Compound | NMR | m/z | SM |
|---|---|---|---|---|
| 24 | 3-(Cyano-dimethyl- | 11.50 (s, br, 1H), 10.45 (s, 1H), | 421 | Method 33 |
| | methyl)-N-[4-methyl-3- | 8.87 (s, 1H), 8.70 (d, 1H), 8.15-7.55 (m, | | and Method |
| | (quinazolin-4-ylamino)- | 8H), 7.40 (d, 1H), 2.20 (s, 3H), | | 24 |
| | phenyl]-benzamide | 1.70 (s, 6H) | | |
| 25 | 3-(Cyano-dimethyl- | 11.35 (s, br, 1H), 10.50 (s, 1H), | 451 | Method 34 |
| | methyl)-N-[3-(6- | 8.80 (s, 1H), 8.20-7.60 (m, 9H), 7.45 (d, | | and Method |
| | methoxy-quinazolin-4- | 1H), 4.00 (s, 3H), 2.20 (s, 3H), | | 24 |
| | ylamino)-4-methyl- | 1.75 (s, 6H) | | |
| | phenyl]-benzamide | | | |
| 26 | 3-(Cyano-dimethyl- | 11.55 (s, br, 1H), 10.47 (s, 1H), | 451 | Method 35 |
| | methyl)-N-[3-(8- | 8.75 (s, 1H), 8.25 (d, 1H), 8.05-7.59 (m, | | and Method |
| | methoxy-quinazolin-4- | 8H), 7.45 (d, 1H), 4.10 (s, 3H), | | 24 |
| | ylamino)-4-methyl- | 2.20 (s, 3H), 1.79 (s, 6H) | | |
| | phenyl]-benzamide | | | |
| 27 | 3-(Cyano-dimethyl- | 11.00 (s, 1H), 10.40 (s, 1H), 8.75 (s, | 451 | Method 36 |
| | methyl)-N-[3-(5- | 1H), 8.00-7.35 (m, 11H), 4.12 (s, 3H), | | and Method |
| | methoxy-quinazolin-4- | 2.20 (s, 3H), 1.75 (s, 6H) | | 24 |
| | ylamino)-4-methyl- | | | |
| | phenyl]-benzamide | | | |
| 28 | 3-(Cyano-dimethyl- | 11.86 (s, br, 1H), 10.50 (s, 1H), | 489 | Method 37 |
| | methyl)-N-[4-methyl-3- | 9.10 (d, 1H), 8.90 (s, 1H), 8.30-7.40 (m, | | and Method |
| | (7-trifluoromethyl- | 10H), 2.20 (s, 3H), 1.75 (s, 6H) | | 24 |
| | quinazolin-4-ylamino)- | | | |
| | phenyl]-benzamide | | | |
| 29 | 3-(Cyano-dimethyl- | 11.82 (s, br, 1H), 10.50 (s, 1H), | 439 | Method 38 |
| | methyl)-N-[3-(7-fluoro- | 8.95 (m, 1H), 8.85 (s, 1H), 8.10-7.60 (m, | | and Method |
| | quinazolin-4-ylamino)- | 9H), 7.40 (d, 1H), 2.20 (s, 3H), | | 24 |
| | 4-methyl-phenyl]- | 1.75 (s, 6H) | | |
| | benzamide | | | |
| 30 | N-[3-(7-Nitro- | | 466 | Method 39 |
| | quinazolin-4-ylamino)- | | | and Method |
| | 4-methyl-phenyl]-3- | | | 24 |
| | (cyano-dimethyl- | | | |
| | methyl)-benzamide | | | |
| 31 | N-{4-Methyl-3-[6- | 11.82 (s, br, 1H), 10.50 (s, 1H), | 436 | Method 45 |
| | methylquinazolin-4- | 8.95 (m, 1H), 8.85 (s, 1H), 8.10-7.60 (m, | | and Method |
| | ylamino]phenyl}-3- | 9H), 7.40 (d, 1H), 2.20 (s, 3H), | | 69 |
| | (trifluoromethyl) | 1.02 (s, 3H) | | |
| | benzamide | | | |
| 32 | 3-(Cyano-dimethyl- | 11.82 (s, br, 1H), 10.50 (s, 1H), | 457 | Method 41 |
| | methyl)-N-[3-(7-chloro- | 8.95 (m, 1H), 8.85 (s, 1H), 8.10-7.60 (m, | | and Method |
| | quinazolin-4-ylamino)- | 9H), 7.40 (d, 1H), 2.20 (s, 3H), | | 24 |
| | 4-methyl-phenyl]- | 1.75 (s, 6H) | | |
| | benzamide | | | |
| 33 | 3-(Cyano-dimethyl- | 11.82 (s, br, 1H), 10.50 (s, 1H), | 437 | Method 42 |
| | methyl)-N-[3-(7-methyl- | 8.95 (m, 1H), 8.85 (s, 1H), 8.10-7.60 (m, | | and Method |
| | quinazolin-4-ylamino)- | 9H), 7.40 (d, 1H), 2.22 (s, 3H), | | 24 |
| | 4-methyl-phenyl]- | 2.20 (s, 3H), 1.75 (s, 6H) | | |
| | benzamide | | | |
| 34 | 3-(Cyano-dimethyl- | 10.85 (s, 1H), 10.50 (s, 1H), 8.70 (s, | 481 | Method 43 |
| | methyl)-N-[3-(5,7- | 1H), 8.12-7.95 (m, 3H), 7.80-7.60 (m, | | and Method |
| | dimethoxy-quinazolin-4- | 3H), 7.40 (d, 1H), 6.92 (m, 2H), | | 24 |
| | ylamino)-4-methyl- | 4.20 (s, 3H), 4.00 (s, 3H), 2.20 (s, 3H), | | |
| | phenyl]-benzamide | 1.74 (s, 6H) | | |
| 35 | 3-(Cyano-dimethyl- | 10.41 (s, 1H), 10.15 (s, 1H), 8.69 (s, | 440 | Method 44 |
| | methyl)-N-[3-(5-fluoro- | 1H), 7.90-8.08 (m, 4H), | | and Method |
| | quinazolin-4-ylamino)- | 7.72-7.80 (m, 1H), 7.55-7.70 (m, 4H), 7.36 (d, | | 24 |
| | 4-methyl-phenyl]- | 1H), 2.20 (s, 3H), 1.75 (s, 6H) | | |
| | benzamide | | | |
| 36 | 3-(Cyano-dimethyl- | 11.82 (s, br, 1H), 10.50 (s, 1H), | 501 | Method 40 |
| | methyl)-N-[3-(7-bromo- | 8.95 (m, 1H), 8.85 (s, 1H), 8.10-7.60 (m, | | and Method |
| | quinazolin-4-ylamino)- | 9H), 7.40 (d, 1H), 2.20 (s, 3H), | | 24 |
| | 4-methyl-phenyl]- | 1.75 (s, 6H) | | |
| | benzamide | | | |
| 37 | 3-(1-Cyano-1- | 10.74 (s, 1H), 8.75 (5, 1H), 7.93 (s, | 468 | Method 46 |
| | methylethyl)-N-{3-[(6- | 1H), 7.79-7.45 (m, 5H), 7.19 (s, 1H), | | and Method |
| | hydroxy-7- | 7.06 (d, 1H), 6.84 (d, 1H), 3.84 (s, | | 24 |
| | methoxyquinazolin-4- | 3H), 2.07 (s, 3H), 1.66 (s, 6H) | | |
| | yl)amino]-4- | | | |
| | methylphenyl}- | | | |
| | benzamide | | | |

### Example 38

### tert-Butyl [3-({6-methoxy-4-[(2-methyl-5-{[3-(trifluoromethyl)benzoyl]amino}phenyl)amino]quinazolin-7-yl}oxy)propyl]carbamate

A solution of N-{3-[(7-hydroxy-6-methoxyquinazolin-4-yl)amino]-4-methylphenyl}-3-(trifluoromethyl)benzamide (Example 70; 100 mg, 0.213 mmol), tert-butyl (3-iodopropyl)carbamate (Method 26; 61 mg, 0.213 mmol, 1.2 equiv) and K₂CO₃ (44 mg, 0.320 mmol, 1.5 equiv) in MeCN (2 ml) were heated to 70° C. for 12 h. The reaction was quenched with water and extracted with EtOAc. The organics were dried with NaCl(sat) and Na₂SO₄(s) and then removed under reduced pressure. The resulting solid was purified by reverse phase preparative HPLC (0.1% TFA in MeCN and water); m/z 626.

### Example 39

### 3-(1-Cyano-1-methylethyl)-N-(4-methyl-3-{[7-(piperidin-4-ylmethoxy)quinazolin-4-yl]amino}phenyl)benzamide

A mixture of 4-(4-{5-[3-(cyano-dimethyl-methyl)-benzoylamino]-2-methyl-phenylamino}-quinazolin-7-yloxymethyl)-piperidine-1-carboxylic acid tert-butyl ester (Example 61; 96 mg, 0.152 mmol) in 4M HCl in dioxane (2 ml) was stirred at 25° C. for 1 h. The solvents were removed under reduced pressure and the residue was purified by reverse phase preparative HPLC (0.1% TFA in MeCN and water) to give 75 mg (93%) of a white solid. NMR: 11.40 (s, 1 h), 10.55 (s, 1H), 8.85-8.45 (m, 4H), 8.10-7.39 (m, 9H), 4.20 (d, 2H), 3.40 (m, 2H), 3.00 (m, 2H), 2.20 (m, 4H), 2.00 (m, 2H), 1.80 (s, 6H), 1.62 (m, 2H); m/z 534.

### Examples 40-44

The following compounds were prepared by the procedure of Example 39, using the appropriate hydroxyl as a starting material.

| Ex | Compound | NMR | m/z | SM |
|---|---|---|---|---|
| 40 | 3-(1-Cyano-1- | 11.30 (s, 1H), 10.45 (s, 1H), | 534 | Example 62 |
| | methylethyl)-N-(4- | 8.85-8.80 (m, 2H), 8.60 (m, 2H), 8.02 (s, 1H), | | |
| | methyl-3-{[7-(piperidin- | 7.90 (m, 2h), 7.77 (m, 1H), | | |
| | 3-ylmethoxy)quinazolin- | 7.66-7.50 (m, 3H), 7.40 (d, 1H), 7.30 (s, 1H), | | |
| | 4-yl]amino}phenyl)- | 4.20 (m, 1H), 4.12 (m, 1H), 3.40 (m, | | |
| | benzamide hydrochloride | 1H), 3.30 (m, 1H), 2.88 (m, 2H), | | |
| | | 2.30 (m, 1H), 2.20 (s, 3H), 1.90 (m, 2H), | | |
| | | 1.75 (m, 7H), 1.40 (m, 1H) | | |
| 41 | (R)—N-{3-[7-(Azetidin-2- | 11.20 (s, br, 1H), 10.40 (s, 1H), | 506 | Example 63 |
| | ylmethoxy)-quinazolin-4- | 9.05 (s, br, 2H), 8.80 (s, 1H), 8.65 (d, 1H), | | |
| | ylamino]-4-methyl- | 8.05 (s, 1H), 7.95 (m, 2H), 7.77 (m, | | |
| | phenyl}-3-(cyano- | 1H), 7.60 (m, 3H), 7.40 (d, 1H), | | |
| | dimethyl-methyl)- | 7.30 (s, 1H), 4.85 (m, 1H), 4.58 (m, 1H), | | |
| | benzamide hydrochloride | 4.50 (m, 1H), 4.00 (m, 2H), 2.55 (m, | | |
| | | 2H), 1.75 (s, 6H). | | |
| 42 | 3-(Cyano-dimethyl- | 10.05 (s, br, 1H), 10.25 (s, 1H), | 520 | Example 64 |
| | methyl)-N-{4-methyl-3- | 9.20 (s, br, 1H), 7.79 (s, br, 1H), 7.57 (s, | | |
| | [7-(pyrrolidin-2- | 1H), 7.42 (m, 1H), 7.80 (s, 1H), | | |
| | ylmethoxy)-quinazolin-4- | 7.75 (m, 2H), 7.56 (m, 2H), 7.40-7.30 (m, | | |
| | ylamino]-phenyl}- | 3H), 7.15 (d, 1H), 7.10 (s, 1H), | | |
| | benzamide hydrochloride | 4.30 (m, 1H), 4.15 (m, 1H), 3.81 (m, 2H), | | |
| | | 3.09 (m, 2H), 1.95 (s, 3H), 1.80 (m, | | |
| | | 2H), 1.60 (m, 1H), 1.52 (s, 6H). | | |
| 43 | 3-(Cyano-dimethyl- | 11.25 (s, br, 1H), 10.45 (s, 1H), | 506 | Example 65 |
| | methyl)-N-{4-methyl-3- | 9.30 (s, br, 1H), 9.15 (s, br, 1H), 8.80 (s, | | |
| | [7-(pyrrolidin-3-yloxy)- | 1H), 8.65 (d, 1H), 8.05 (s, 1H), | | |
| | quinazolin-4-ylamino]- | 7.90 (m, 2H), 7.76 (d, 1H), 7.60 (m, 2H), | | |
| | phenyl}-benzamide | 7.51 (d, 1H), 7.40 (d, 1H), 7.35 (s, | | |
| | hydrochloride | 1H), 5.40 (m, 1H), 3.50 (m, 2H), | | |
| | | 3.35 (m, 2H), 2.37 (m, 1H), 2.25 (m, 1H), | | |
| | | 2.19 (s, 3H), 1.75 (s, 6H). | | |
| 44 | N-{3-[7-(Azetidin-3- | 11.35 (s, 1H), 10.45 (s, 1H), 8.90 (s, | 506 | Example 66 |
| | ylmethoxy)-quinazolin-4- | br, 2H), 8.80 (s, 1H), 8.67 (d, 1H), | | |
| | ylamino]-4-methyl- | 8.05-7.35 (m, 9H), 4.40 (d, 2H), | | |
| | phenyl}-3-(cyano- | 4.17 (m, 2H), 3.96 (m, 2H), 3.31 (m, 1H), | | |
| | dimethyl-methyl)- | 2.20 (s, 3H), 1.76 (s, 6H). | | |
| | benzamide hydrochloride | | | |

### Example 45

### 3-(Cyano-dimethyl-methyl)-N-(3-{7-[2-(2-hydroxymethyl-pyrrolidin-1-yl)-ethoxy]-quinazolin-4-ylamino}-4-methyl-phenoyl)-benzamide hydrochloride

A mixture of N-{3-[7-(2-bromo-ethoxy)-quinazolin-4-ylamino]-4-methyl-phenyl}-3-(cyano-dimethyl-methyl)-benzamide (Example 58; 97 mg, 0.178 mmol), pyrrolidin-2-yl-methanol (20 mg, 0.196 mmol, 1.1 eq) and K₂CO₃ (123 mg, 0.89 mmol, 5 eq) in MeCN (10 ml) was refluxed for 12 h. The heterogeneous mixture was filtered and the solids were washed with MeOH. The organics were concentrated under reduced pressure and the residue was purified by reverse phase preparative HPLC (0.1% TFA in MeCN and water) to yield 50 mg of (50%) a white solid. NMR: 11.70 (s, 1H), 10.55 (s, 1H), 10.42 (s, br, 1H), 8.90 (d, 1H), 8.80 (s, 1H), 8.10 (s, 1H), 7.97 (d, 1H), 7.80 (s, 1H), 7.60 (m, 2H), 7.45 (s, 1H), 7.35 (d, 1H), 4.70 (m, 1H), 4.60 (m, 1H), 3.95 (m, 1H), 3.75 (m, 2H), 3.60 (m, 2H), 3.25 (m, 2H), 2.20 (s, 3H), 2.15-1.90 (m, 3H), 1.75 (m, 7H); m/z 564.

### Example 46

### N-{3-[7-(3-Bromo-propoxy)-quinazolin-4-ylamino]-4-methyl-phenyl}-3-(cyano-dimethyl-methyl)-benzamide

A mixture of 3-(cyano-dimethyl-methyl)-N-[3-(7-hydroxy-quinazolin-4-ylamino)-4-methyl-phenyl]-benzamide (Example 68; 300 mg, 0.686 mmol), 1,3-dibromopropane (277 mg, 1.372 mmol) and K₂CO₃ (189 mg, 1.372 mmol) in acetone-1,4-dioxane-DMF (5;1;1; 10 ml) was refluxed for 12 h. The heterogeneous mixture was filtered and the solids were washed with MeOH. The organics were concentrated under reduced pressure and the residue was purified by column chromatography utilizing an ISCO system (hexane-EtOAc) to yield 112 mg (29%) of a light yellow solid. m/z 558.

### Examples 47-52

The following compounds were prepared by the procedure of Example 46, using Example 68 (Examples 47-51) and Example 37 (Example 52) and the appropriate alkyl halide as a starting material.

| Ex | Compound | NMR | m/z | SM |
|---|---|---|---|---|
| 47 | 3-(Cyano-dimethyl- | 11.57 (s, 1H), 10.55 (s, 1H), 8.87 (s, | 479 | 1-Bromo- |
| | methyl)-N-[4-methyl- | 1H), 8.75 (d, 1H), 8.10 (s, 1H), 8.00 (d, | | propane |
| | 3-(7-propoxy- | 1H), 7.95 (s, 1H), 7.80 (d, 1H), 7.72 (d, | | |
| | quinazolin-4- | 1H), 7.65 (m, 1H), 7.68 (d, 1H), | | |
| | ylamino)-phenyl]- | 7.45 (d, 1H), 7.35 (s, 1H), 4.25 (t, 2H), | | |
| | benzamide | 2.25 (s, 3H), 1.90 (m, 2H), 1.80 (s, 6H), | | |
| | | 1.10 (t, 3H) | | |
| 48 | 3-(1-Cyano-1- | 11.42 (s, 1H), 10.45 (s, 1H), 8.77 (s, | 479 | 2-Bromo- |
| | methylethyl)-N-{3- | 1H), 8.65 (d, 1H), 8.06 (s, 1H), 7.92 (m, | | propane |
| | [(7- | 1h), 7.90 (s, 1H), 7.85 (d, 1H), 7.68 (d, | | |
| | isopropoxyquinazolin- | 1H), 7.60 (m, 1H), 7.50 (d, 1H), | | |
| | 4-yl)amino]-4-methyl- | 7.40 (d, 1H), 7.30 (s, 1H), 4.90 (m, 1H), | | |
| | phenyl}benzamide | 2.20 (s, 3H), 1.76 (s, 6H), 1.40 (d, 6H) | | |
| 49 | 3-(Cyano-dimethyl- | NMR: 11.65 (s, 1H), 10.60 (s, br, 1H), | 522. | 3-Chloro- |
| | methyl)-N-{3-[7-(3- | 10.50 (s, 1H), 8.80 (m, 2H), 8.07 (s, | | propyl- |
| | dimethylamino- | 1H), 7.95 (d, 1H), 7.90 (s, 1H), | | dimethyl- |
| | propoxy)-quinazolin- | 7.75-7.50 (m, 4H), 7.36 (m, 2H), 4.30 (m, | | amine |
| | 4-ylamino]-4-methyl- | 2H), 3.27 (m, 2H), 2.80 (d, 6H), | | hydro- |
| | phenyl}-benzamide | 2.25 (m, 2H), 2.20 (s, 3H), 1.75 (s, 6H) | | chloride |
| 50 | 3-(1-Cyano-1- | NMR: 8.7 (s, 1H), 8.43 (d, 1H), | 495 | 1-Chloro- |
| | methylethyl)-N-(3- | 6.84-7.93 (m, 8H), 4.42 (m, 2H), 3.76 (m, | | 2-methoxy- |
| | {[7-(2- | 2H), 3.41 (s, 3H), 2.07 (s, 3H), 1.66 (s, | | ethane |
| | methoxyethoxy)- | 6H) | | |
| | quinazolin-4-yl]- | | | |
| | amino}-4-methyl- | | | |
| | phenyl)benzamide | | | |
| 51 | N-{3-[7-(2-Bromo- | 10.36 (s, 1H), 9.65 (s, 1H), 8.46 (d, | 544 | 1,2- |
| | ethoxy)-quinazolin-4- | 1H), 8.40 (s, 1H), 8.10-7.60 (m, 6H), | | dibromo- |
| | ylamino]-4-methyl- | 7.35-7.25 (m, 3H), 4.56 (t, 2H), 3.92 (t, | | ethane |
| | phenyl}-3-(cyano- | 2H), 2.20 (s, 3H), 1.80 (s, 6H) | | |
| | dimethyl-methyl)- | | | |
| | benzamide | | | |
| 52 | 3-(1-Cyano-1- | 8.7 (s, 1H), 6.8-7.93 (m, 9H), 4.14 (m, | 538 | 3-Chloro- |
| | methylethyl)-N-(3-{6- | 2H), 3.96 (s, 3H), 2.98 (m, 2H), 2.45 (s, | | propyl- |
| | [3-(dimethylamino)- | 6H), 2.07 (s, 3H), 1.66 (s, 6H) | | dimethyl- |
| | propoxy]-7-methoxy- | | | amine |
| | quinazolin-4-yl}-4- | | | hydro- |
| | methylphenyl)- | | | chloride |
| | benzamide | | | |

### Example 53

### tert-Butyl [3-({4-[(5-{[3-(1-cyano-1-methylethyl)benzoyl]amino}-2-methylphenyl)amino]quinazolin-7-yl}oxy)propyl]carbamate

A mixture of 3-(cyano-dimethyl-methyl)-N-[3-(7-hydroxy-quinazolin-4-ylamino)-4-methyl-phenyl]-benzamide (Example 68; 100 mg, 0.229 mmol), (3-bromo-propyl)-carbamic acid tert-butyl ester (109 mg, 0.458 mmol) and K₂CO₃ (126 mg, 0.916 mmol) in acetone-1,4-dioxane-DMF (5:1:1; 10 ml) was refluxed for 12 h. The heterogeneous mixture was filtered and the solids were washed with MeOH. The organics were concentrated under reduced pressure and the residue was purified by column chromatography utilizing an ISCO system (hexane-EtOAc) to yield 90 mg (66%) of the title compound; m/z 594.

### Example 54

### 4-Dimethylaminomethyl-N-[3-(7-methoxy-quinazolin-4-ylamino)-4-methyl-phenyl]-3-trifluoromethyl-benzamide

A mixture of N³-(7-methoxy-quinazolin-4-yl)-4-methyl-benzene-1,3-diamine (Method 62; 80 mg, 0.286 mmol), 4-dimethylaminomethyl-3-trifluoromethyl-benzoic acid (Method 48; 71 mg, 0.286 mmol), HATU (130 mg, 0.343 mmol) and DIEA (147 mg, 1.1 mmol) DMF (2 ml) was stirred at 25° C. for 2 h. The reaction mixture was purified by reverse phase preparative HPLC (0.1% TFA in MeCN and water) to yield 85 mg (58%) of a white solid. NMR: 11.55 (s, 1H), 11.00 (s, br, 1H), 10.70 (s, 1H), 8.71 (m, 2H), 8.33 (m, 3H), 7.85 (s, 1H), 7.65 (d, 1H), 7.45-7.28 (m, 3H), 4.50 (s, 2H), 3.92 (s, 6H), 2.72 (s, 3H), 2.10 (s, 3H); m/z 509.

### Example 55

### 2-(Cyano-dimethyl-methyl)-N-[3-(7-methoxy-quinazolin-4-ylamino)-4-methyl-phenyl]-isonicotinamide

A mixture of N³-(7-methoxy-quinazolin-4-yl)-4-methyl-benzene-1,3-diamine (Method 62; 81 mg, 0.289 mmol), 2-(1-cyano-1-methylethyl)isonicotinic acid (Method 60; 55 mg, 0.289 mmol), HATU (132 mg, 0.347 mmol) and DIEA (147 mg, 1.1 mmol) in DMF (2 ml) was stirred at 25° C. for 2 h. The organics were removed under reduced pressure and the crude reaction mixture was purified by reverse phase preparative HPLC (0.1% TFA in MeCN and water) to yield 45 mg (34%) of a yellow solid. NMR: 11.46 (s, 1H), 10.70 (s, 1H), 8.80 (m, 2H), 8.70 (d, 1H), 7.90 (s, 1H), 7.85 (m, 2H), 7.50 (d, 1H), 7.40 (d, 1H), 7.22 (s, 1H), 4.00 (s, 3H), 2.15 (s, 3H), 1.80 (s, 6H); m/z 452.

### Example 56

The following compound was prepared by the procedure of Example 55, using the appropriate starting materials.

| Ex | Compound | NMR | m/z | SM |
|---|---|---|---|---|
| 56 | 1-tert-Butyl-N-{3-[(7- | 11.71 (s, 1H), 10.70 (s, 1H), 8.80 (m, | 444 | Method 62 |
| | methoxyquinazolin-4- | 2H), 7.80 (s, 1H), 7.55 (m, 2H), 7.35 (m, | | and 2-tert- |
| | yl)amino]-4- | 2H), 6.40 (s, 1H), 4.00 (s, 3H), 2.15 (s, | | butyl-5- |
| | methylphenyl}-3- | 3H), 2.12 (s, 3H), 1.55 (s, 9H) | | methyl-2H- |
| | methyl-1H-pyrazole-5- | | | pyrazole-3- |
| | carboxamide | | | carboxylic |
| | | | | acid |

### Example 57

### 3-(Cyano-dimethyl-methyl)-5-fluoro-N-[3-(7-methoxy-quinazolin-4-ylamino)-4-methyl-phenyl]-benzamide

A mixture of 4-chloro-7-methoxy-quinazoline (Method 32; 700 mg, 3.6 mmol) and N-(3-amino-4-methyl-phenyl)-3-(cyano-dimethyl-methyl)-5-fluoro-benzamide (Method 5; 900 mg, 2.89 mmol) in isopropanol (30 ml) was refluxed for 4 h. The organics were removed under reduced pressure and the residue was purified by column chromatography utilizing an ISCO system (EtOAc) and then purified by reverse phase preparative HPLC (0.1% TFA in MeCN and water) to give 1.1 g (81%) of a light yellow solid. NMR: 11.48 (s, 1H), 10.55 (s, 1H), 8.80 (s, 1H), 8.70 (d, 1H), 7.95 (s, 1H), 7.90 (s, 1H), 7.80 (d, 1H), 7.66 (m, 2H), 7.50 (d, 1H), 7.48 (d, 1H), 7.30 (m, 1H), 4.00 (s, 3H), 2.20 (s, 3H), 1.78 (s, 6H); m/z 469.

### Example 58

### N-{3-[7-(2-Bromo-ethoxy)-quinazolin-4-ylamino]-4-methyl-phenyl}-3-(cyano-dimethyl-methyl)-benzamide

A mixture of 3-(cyano-dimethyl-methyl)-N-[3-(7-hydroxy-quinazolin-4-ylamino)-4-methyl-phenyl]-benzamide (Example 68; 100 mg, 0.229 mmol), 1,2-dibromoethane (86 mg, 0.458 mmol) and K₂CO₃ (63 mg, 0.458 mmol) in acetone-1,4-dioxane-DMF (5:1:1; 10 ml) was refluxed for 12 h. The heterogeneous mixture was filtered and the solids were washed with MeOH. The organics were concentrated under reduced pressure and the residue was purified by column chromatography utilizing an ISCO system (hexane-EtOAc) to yield 97 mg (78%) of a light yellow solid. NMR: 10.36 (s, 1H), 9.65 (s, 1H), 8.46 (d, 1H), 8.40 (s, 1H), 8.10-7.60 (m, 6H), 7.35-7.25 (m, 3H), 4.56 (t, 2H), 3.92 (t, 2H), 2.20 (s, 3H), 1.80 (s, 6H); m/z 544.

### Example 59

### 3-(Cyano-dimethyl-methyl)-N-(3-{7-[3-(2-hydroxymethyl-pyrrolidin-1-yl)-propoxy]-quinazolin-4-ylamino}-4-methyl-phenyl)-benzamide hydrochloride

A mixture of N-{3-[7-(3-bromo-propoxy)-quinazolin-4-ylamino]-4-methyl-phenyl}-3-(cyano-dimethyl-methyl)-benzamide (Example 46; 112 mg, 0.2 mmol), pyrrolidin-2-yl-methanol (40 mg, 0.4 mmol) and K₂CO₃ (138 mg, 1 mmol) in MeCN (10 ml) was refluxed for 12 h. The heterogeneous mixture was filtered and the solids were washed with MeOH. The organics were concentrated under reduced pressure and the residue was purified by reverse phase preparative HPLC (0.1% TFA in MeCN and water) to yield 75 mg (65%) of a white solid. NMR: 11.50 (s, br, 1H), 10.50 (s, 1H), 9.85 (s, br, 1H), 8.80-8.75 (m, 2H), 8.05-7.90 (m, 3H), 7.75-7.50 (m, 4 h), 7.38-7.31 (m, 2H), 4.35 (m, 2H), 3.80-3.15 (m, 8H), 2.20 (m, 2H), 2.18 (s, 3H), 2.10-1.90 (m, 3H), 1.70 (m, 7H); m/z 578.

### Example 60

### N-{3-[7-(3-Amino-propoxy)-quinazolin-4-ylamino]-4-methyl-phenyl}-3-(cyano-dimethyl-methyl)-benzamide hydrochloride

A mixture of tert-butyl [3-({4-[(5-{[3-(1-cyano-1-methylethyl)benzoyl]amino}-2-methylphenyl)amino]quinazolin-7-yl}oxy)propyl]carbamate (Example 53; 90 mg, 0.152 mmol) in 4M HCl in dioxane (2 ml) was stirred at 25° C. for 1 h. The organics were removed under reduced pressure and the residue was purified by reverse phase preparative HPLC (0.1% TFA in MeCN and water) to give 68 mg (91%) of a white solid. NMR: 11.12 (s, br, 1H), 10.35 (s, 1H), 8.70 (s, 1H), 8.50 (d, 1H), 7.95-7.16 (m, 12H), 4.20 (m, 2H), 2.95 (m, 2H), 2.10 (s, 3H), 2.05 (m, 2H), 1.66 (s, 6H); m/z 494.

### Example 61

### 4-(4-{5-[3-(Cyano-dimethyl-methyl)-benzoylamino]-2-methyl-phenylamino}-quinazolin-7-yloxymethyl)-piperidine-1-carboxylic acid tert-butyl ester

A mixture of 3-(cyano-dimethyl-methyl)-N-[3-(7-hydroxy-quinazolin-4-ylamino)-4-methyl-phenyl]-benzamide (Example 68; 150 mg, 0.343 mmol), 4-hydroxymethyl-piperidine-1-carboxylic acid tert-butyl ester (147 mg, 0.686 mmol), azodicarboxylic acid diethylester (40% in toluene; 1.72 mmol, 5 equiv) and triphenyl phosphine (451 mg, 1.72 mmol, 5 equiv) in THF (10 ml) was stirred at 25° C. for 12 h. The solvents were removed under reduced pressure. The residue was purified first by column chromatography utilizing an ISCO system (hexane-EtOAc) and then by reverse phase preparative HPLC (0.1% TFA in MeCN and water) to give 96 mg (44%) of a light yellow solid. NMR: 8.35 (m, 2H), 8.02 (s, 1H), 7.95 (s, 1H), 7.80 (d, 1H), 7.69 (m, 2H), 7.56-7.20 (m, 6H), 4.20 (m, 2H), 4.00 (d, 2H), 2.80 (m, 2H), 2.20 (s, 3H), 2.05 (m, 1H), 1.85 (m, 2H), 1.75 (s, 3H), 1.49 (s, 9H), 1.30 (m, 2H); m/z 634.

### Examples 62-67

The following compounds were prepared by the procedure of Example 61 using the appropriate intermediates.

| Ex. | Compound | M/z | SM |
|---|---|---|---|
| 62 | 3-(4-{5-[3-(Cyano-dimethyl-methyl)-benzoylamino]- | 634 | 3-Hydroxymethyl- |
| | 2-methyl-phenylamino}-quinazolin-7-yloxymethyl)- | | piperidine-1- |
| | piperidine-1-carboxylic acid tert-butyl ester | | carboxylic acid tert- |
| | | | butyl ester |
| 63 | (R)-2-(4-{5-[3-(Cyano-dimethyl-methyl)- | 606 | R-2-Hydroxymethyl- |
| | benzoylamino]-2-methyl-phenylamino}-quinazolin- | | azetidine-1-carboxylic |
| | 7-yloxymethyl)-azetidine-1-carboxylic acid tert-butyl | | acid tert-butyl ester |
| | ester | | |
| 64 | 2-(4-{5-[3-(Cyano-dimethyl-methyl)-benzoylamino]- | 620 | 2-Hydroxymethyl- |
| | 2-methyl-phenylamino}-quinazolin-7-yloxymethyl)- | | pyrrolidine-1- |
| | pyrrolidine-1-carboxylic acid tert-butyl ester | | carboxylic acid tert- |
| | | | butyl ester |
| 65 | 3-(4-{5-[3-(Cyano-dimethyl-methyl)-benzoylamino]- | 606 | 3-Hydroxy- |
| | 2-methyl-phenylamino}-quinazolin-7-yloxy)- | | pyrrolidine-1- |
| | pyrrolidine-1-carboxylic acid tert-butyl ester | | carboxylic acid tert |
| | | | butyl ester |
| 66 | 3-(4-{5-[3-(Cyano-dimethyl-methyl)-benzoylamino]- | 606 | 3-Hydroxymethyl- |
| | 2-methyl-phenylamino}-quinazolin-7-yloxymethyl)- | | azetidine-1-carboxylic |
| | azetidine-1-carboxylic acid tert-butyl ester | | acid tert-butyl ester |
| 67 | (S)-3-(Cyano-dimethyl-methyl)-N-{4-methyl-3-[7- | 534 | (S)-(1-methyl- |
| | (1-methyl-pyrrolidin-2-ylmethoxy)-quinazolin-4- | | pyrrolidin-2-yl)- |
| | ylamino]-phenyl}-benzamide hydrochloride¹ | | methanol |

¹NMR: 11.67 (s, 1H), 11.00 (s, br, 1H), 10.55 (s, 1H), 8.86 (m, 2H), 8.10-7.95 (m, 3H), 7.80-7.62 (m, 4H), 7.42 (m, 2H), 4.67 (m, 2H), 4.00 (m, 1H), 3.45 (m, 1H), 3.05 (s, 3H), 2.40 (m, 2H), 2.25 (s, 3H), 2.13 (m, 1H), 2.07 (m, 1H), 1.96 (m, 1H), 1.80 (s, 6H).

### Example 68

### 3-(Cyano-dimethyl-methyl)-N-[3-(7-hydroxy-quinazolin-4-ylamino)-4-methyl-phenyl]-benzamide

A suspension of N-[3-(7-benzyloxy-quinazolin-4-ylamino)-4-methyl-phenyl]-3-(cyano-dimethyl-methyl)-benzamide (Example 21; 3.13 g, 5.94 mmol) and 10% Pd/C (400 mg) in MeOH (150 ml) was stirred at 25° C. under a hydrogen atmosphere. The reaction mixture was filtered through diatomaceous earth and the organics were concentrated under reduced pressure to give 2.6 g (99%) of a light yellow solid. NMR: 10.41 (s, 1H), 10.30 (s, 1H), 9.46 (s, 1H), 8.33 (d, 1H), 8.27 (s, 1H), 8.05 (s, 1H). 7.90 (d, 1H), 7.75 (m, 2H), 7.60 (m, 2H), 7.30 (d, 1H), 7.10 (d, 1H), 7.01 (s, 1H), 2.15 (s, 3H), 1.75 (s, 6H); m/z 437.

### Example 69

### N-[3-(7-Amino-quinazolin-4-ylamino)-4-methyl-phenyl]-3-(cyano-dimethyl-methyl)-benzamide

A mixture of 3-(cyano-dimethyl-methyl)-N-[3-(7-nitro-quinazolin-4-ylamino)-4-methyl-phenyl]-benzamide (Example 30; 1.18 g, 2.05 mmol) and 10% Pd/C (100 mg) in MeOH (50 ml) was stirred at 25° C. under a hydrogen atmosphere for 3 h. The reaction mixture was filtered through a bed of diatomaceous earth and the organics were concentrated under reduced pressure. The residue was purified by column chromatography utilizing an ISCO system (EtOAc-DCM-MeOH) to yield 800 mg (90%) of the desired product. NMR 10.60 (s, 1H), 10.45 (s, 1H), 8.48 (s, 1H), 8.35 (d, 1H), 8.10 (s, 1H), 7.95-7.60 (m, 5H), 7.31 (d, 1H), 7.00-6.65 (m, 4H), 2.16 (s, 3H), 1.75 (s, 6H).

### Example 70

### N-{3-[(7-Hydroxy-6-methoxyquinazolin-4-yl)amino]-4-methylphenyl}-3-(trifluoromethyl)benzamide

A solution of 4-[(5-amino-2-methylphenyl)amino]-6-methoxyquinazolin-7-ol (Method 6; 900 mg, 3.04 mmol) and triethylamine (1.27 mL, 9.12 mmol, 3.0 equiv) in DCM (10 mL) was treated with 3-(trifluoromethyl)benzyl chloride (0.55 mL, 3.64 mmol, 1.2 equiv) at 25° C. for 12 h. The reaction was quenched with water and extracted with EtOAc. The organics were dried with NaCl(sat) and Na₂SO₄(s) and then removed under reduced pressure. The resulting residue was purified by column chromatography utilizing an ISCO system (DCM-MeOH) to give 0.43 g (30%); m/z 469.

### Preparation of Starting Materials

### Method 1

### 4-Oxo-3,4-dihydroquinazoline-7-carboxylic acid

A mixture of 2-aminoterephthalic acid (6.90 g, 0.038 mol) and formamide (14 ml) was heated to 180° C. for 12 h. The reaction was allowed to cool and acetone was added. The resulting precipitate was collected by vacuum filtration (4.38 g, 60%); m/z 191.

### Method 2

### 4-Chloroquinazoline-7-carboxylic acid

A mixture of 4-oxo-3,4-dihydroquinazoline-7-carboxylic acid (Method 1; 1.00 g, 5.26 mmol), oxalyl chloride (1.37 ml, 15.8 mmol, 3.0 equiv) in DCM (15 ml) was treated with DMF (0.1 ml). The reaction mixture was stirred under Ar for 3 h at 25° C. The solvents were removed under reduced pressure; m/z 209.

### Method 3

### 4-[(2-Methyl-5-nitrophenylamino]quinazoline-7-carboxylic acid

A mixture of 4-chloroquinazoline-7-carboxylic acid (Method 2; 1.10 g, 5.26 mmol) and 2-methyl-5-nitroaniline (960 mg, 6.31 mmol, 1.2 equiv) in DCM (15 ml) was treated with iPr₂NEt (1.4 ml, 7.89 mmol, 1.5 equiv). The reaction mixture was stirred under Ar for 12 h at 25° C. The resulting precipitate was collected by vacuum filtration; m/z 325.

### Method 4

The following compound was prepared by the procedure of Method 3 using appropriate SMs.

| Meth | Compound | m/z | SM |
|---|---|---|---|
| 4 | 6,7-Dimethoxy-N-(2-methyl-5- | 341 | 2-methyl-5-nitroaniline |
| | nitrophenyl)quinazolin-4-amine | | and 4-chloro-6,7- |
| | | | dimethoxy-quinazoline |

### Method 5

### N-(3-Amino-4-methyl-phenyl)-3-(cyano-dimethyl-methyl)-5-fluoro-benzamide

A mixture of 3-(cyano-dimethyl-methyl)-5-fluoro-N-(4-methyl-3-nitro-phenyl)-benzamide (Method 21; 2.5 g, 7.33 mmol) and 10% Pd/C (200 mg) in MeOH (150 ml) was treated with a hydrogen atmosphere for 48 h at 25° C. The reaction mixture was filtered through diatomaceous earth and the organics were concentrated under reduced pressure. The residue was purified by column chromatography utilizing an ISCO system (hexane-EtOAc), to yield 900 mg (39.4%) of a white solid. NMR: 7.90 (s, 1H), 7.70 (s, 1H), 7.40 (d, 1 h), 7.30 (d, 1H), 7.20 (s, 1H), 6.92 (d, 1H), 6.65 (d, 1H), 3.30 (s, 2H), 2.10 (s, 3H), 1.70 (s, 6H); m/z 311.

### Method 6

The following compound was prepared by the procedure of Method 5, using the appropriate SMs.

| Meth | Compound | m/z | SM |
|---|---|---|---|
| 6 | 4-[(5-Amino-2-methylphenyl)amino]- | 202 | Method 20 |
| | 6-methoxyquinazolin-7-ol | | |

### Method 7

### 4-[(5-Amino-2-methylphenyl)amino]quinazoline-7-carboxylic acid

4-[(2-Methyl-5-nitrophenyl)amino]quinazoline-7-carboxylic acid (Method 3; 1.71 g, 5.26 mmol) and 30% Pd/C (200 mg) in MeOH (30 ml) were shaken in a Parr hydrogenator under 45 psi hydrogen for 3 h. The reaction mixture was filtered through diatomaceous earth, and the resulting filtrate was concentrated under reduced pressure giving the desired compound; m/z 295.

### Method 8

The following compound was prepared by the procedure of Method 7 using the appropriate SM.

| Meth | Compound | m/z | SM |
|---|---|---|---|
| 8 | N³-(6,7-Dimethoxyquinazolin-4-yl)-4- | 311 | Method 4 |
| | methylbenzene-1,3-diamine | | |

### Method 9

### 3-Cyanomethyl-benzoic acid methyl ester

A suspension of methyl-3-(bromomethyl)benzoate (13.5 g, 58.9 mmol) and sodium cyanide (4.33 g, 88.4 mmol) in DMF (25 ml) and water (1 ml) was stirred at 75° C. for 5 h. The reaction mixture was quenched with water and extracted with EtOAc. The combined organics were dried and concentrated under reduced pressure. The resulting residue was purified by column chromatography utilizing an ISCO system (hexane-EtOAc) to give 7.2 g (70%) of colourless oil. NMR: 7.90 (s, 1H), 7.86 (d, 1H), 7.60 (d, 1H), 7.50 (m, 1H), 4.10 (s, 2H), 3.80 (s, 3H); m/z 175.

### Method 10

The following compound was prepared by the procedure of Method 9 using the appropriate SM.

| Meth | Compound | m/z | SM |
|---|---|---|---|
| 10 | (2-Fluoro-3-methylphenyl)acetonitrile | 150 | 1-(Bromomethyl)- |
| | | | 2-fluoro-3- |
| | | | methylbenzene |

### Method 11

### 3-(1-Cyano-1-methylethyl)benzoic acid methyl ester

A solution of 3-cyanomethyl-benzoic acid methyl ester (Method 9; 7.2 g, 41.1 mmol) in DMSO (80 ml) was treated with sodium hydride (60%, 4.9 g, 123.3 mmol, 3 equiv). Methyl iodide was added dropwise at 0° C. The reaction mixture was stirred at 25° C. for 12 h. The reaction mixture was quenched with water and extracted with EtOAc. The combined organics were dried and concentrated under reduced pressure. The crude product was purified by column chromatography utilizing an ISCO system (hexane-EtOAc) to give 5.5 g (66%) of a colourless oil. NMR: 8.05 (s, 1H), 7.90 (d, 1H), 7.75 (d, 1H), 7.55 (m, 1H), 3.80 (s, 3H), 1.62 (s, 6H); m/z 203.

### Methods 12-15

The following compounds were prepared by the procedure of Method 11, using the appropriate SMs.

| Meth | Compound | m/z | SM |
|---|---|---|---|
| 12 | Methyl 3-(4-cyanotetrahydro- | 246 | Method 9 and 1-bromo-2- |
| | 2H-pyran-4-yl)benzoate | | (2-bromoethoxy)ethane |
| 13 | Methyl 3-(1- | 202 | Method 9 and 1,2- |
| | cyanocyclopropyl)benzoate | | dibromoethane |
| 14 | Methyl 3-(1-cyanocyclo- | 216 | Method 9 and 1,3- |
| | butyl)benzoate | | dibromopropane |
| 15 | 2-(2-Fluoro-3-methylphenyl)- | 178 | Method 10 |
| | 2-methylpropanenitrile | | |

### Method 16

### 3-(1-Cyano-1-methylethyl)benzoic acid

A solution of 3-(1-cyano-1-methylethyl)benzoic acid methyl ester (Method 11; 5.5 g, 27.1 mmol) in 100 ml of THF-MeOH—H₂O (3:1:1) was treated with lithium hydroxide (1.95 g) in water (20 ml). The mixture was stirred at 25° C. for 12 h. The organics were removed under reduced pressure and the residue was diluted with water, and then acidified with 10% HCl to pH=1-3. The resulting white solid (4.83 g, 94%) was collected by vacuum filtration. NMR: 13.00 (s, 1H), 7.95 (s, 1H), 7.80 (d, 1H), 7.65 (d, 1H), 7.45 (m, 1H), 1.60 (s, 6H); m/z 189.

### Methods 17-19

The following compounds were prepared by the procedure of Method 16 using the appropriate SMs.

| Meth | Compound | m/z | SM |
|---|---|---|---|
| 17 | 3-(1-Cyanocyclobutyl)benzoic acid | 202 | Method 14 |
| 18 | 3-(4-Cyanotetrahydro-2H-pyran-4-yl)benzoic | 232 | Method 12 |
| | acid | | |
| 19 | 3-(1-Cyanocyclopropyl)benzoic acid | 188 | Method 13 |

### Method 20

### 6-Methoxy-4-[(2-methyl-5-nitrophenyl)amino]quinazolin-7-ol

A solution of 7-benzyloxy-4-chloro-6-methoxyquinazoline (2.00 g, 6.65 mmol) and 2-methyl-5-nitroaniline (1.01 g, 6.65 mmol) in EtOH (20 ml) was heated to 95° C. for 12 h. The organics were removed under reduced pressure. The resulting solid was utilized without further purification; m/z 417.

### Method 21

### 3-(Cyano-dimethyl-methyl)-5-fluoro-N-(4-methyl-3-nitro-phenyl)-benzamide

A mixture of 4-methyl-3-nitro-phenylamine (1.6 g, 10.6 mmol), 3-(cyano-dimethyl-methyl)-5-fluoro-benzoic acid (Method 55; 2.2 g, 10.6 mmol), HATU (4.8 g, 12.7 mmol) and DIEA (4.1 g, 31.8 mmol) in DMF (15 mL) was stirred at 25° C. for 3 h. Water was added and the reaction mixture was extracted with EtOAc. The organics were concentrated under reduced pressure, and the residue was purified by column chromatography utilizing an ISCO system (hexane-EtOAc) to yield 2.5 g (69%) of a yellow solid. NMR: 8.30 (s, 1H), 8.00 (s, 1H), 7.90 (d, 1H), 7.85 (s, 1H), 7.60 (d, 1H), 7.40 (m, 2H), 2.65 (s, 3H), 1.80 (s, 6H); m/z 341.

### Method 22

### 3-Isopropylbenzoic acid

1-Bromo3-isopropylbenzene (500 mg, 2.51 mmol) in pentane-ether (1:1) (8 ml) at −78° C. under Ar was treated with t-BuLi (1.7 M in pentane, 5.02 mmol, 2.0 equiv). The reaction stirred for 15 min and then CO_{2(g)} was bubbled through the reaction mixture. After 10 min, the reaction was quenched with 10% NaOH and extracted with EtOAc. The aqueous layer was acidified with 10% HCl and extracted with EtOAc. The organics were dried with NaCl(sat) and Na₂SO₄(s) and then removed under reduced pressure; m/z 165.

### Method 23

### 3-(1-Cyano-1-methylethyl)-N-(4-methyl-3-nitro-phenyl)benzamide

A mixture of 4-methyl-3-nitroaniline (2.74 g, 18 mmol), 3-(1-cyano-1-methylethyl) benzoic acid (Method 16; 3.4 g, 18 mmol), EDCI (6.9 g, 36 mmol), HOBt (2.43 g, 18 mmol) and diisopropylethyl amine (3.48 g, 27 mmol) in DMF (30 ml) was stirred at 25° C. for 12 h. The reaction mixture was diluted with DCM and washed with water. The organic phase was dried with NaCl(sat) and Na₂SO₄(s). The solvent was removed under reduced pressure and the resulting product was purified by column chromatography utilizing an ISCO system (hexane-EtOAc) to give 4.4 g (53%). NMR: 10.50 (s, 1H), 8.40 (s, 1H), 7.40-7.95 (m, 6H), 3.20 (s, 3H), 1.65 (s, 6H); m/z 323.

### Method 24

### N-(3-Amino-4-methylphenyl)-3-(1-cyano-1-methylethyl benzamide

A suspension of 3-(1-cyano-1-methylethyl)-N-(4-methyl-3-nitro-phenyl)benzamide (Method 23; 4 g, 13.9 mmol) and 5% Pd/C (400 mg) in hydrazine hydrate (100 ml) and ethanol (100 ml) was heated to reflux for 3 h and then stirred at 80° C. for 12 h. The reaction mixture was filtered through diatomaceous earth and the organics were removed under reduced pressure. The residue was purified by column chromatography using an ISCO system (hexane-EtOAc) to give 3.7 g (91%) of an orange gum. NMR: 9.95 (s, 1H), 8.00 (s, 1H), 7.90 (d, 1H), 7.70 (d, 1H), 7.55 (m, 1H), 7.05 (s, 1H), 6.80-6.87 (m, 2H), 4.85 (s, 2H), 2.05 (s, 3H), 1.85 (s, 6H); m/z 293.

### Method 25

### 3-[(Dimethylamino)sulfonyl]benzoic acid

A solution of 3-(chlorosulfonyl)benzoic acid (2.60 g, 12 mmol) in DCM (20 ml) was treated with dimethylamine (2.0 M in THF, 20 ml, 40 mmol, 3.3 equiv). After 30 min, the reaction was quenched with 10% HCl and extracted with EtOAc. The organics were washed with NaCl(sat) and dried with Na₂SO₄(s). The organics were then removed under reduced pressure to give 1.80 g, 65%; m/z 229.

### Method 26

### tert-Butyl (3-iodopropyl)carbamate

Triphenylphosphine (11.21 g, 43 mmol) and imidazole (2.91 g, 43 mmol, 1.5 equiv) in DCM at 0° C. under Ar was treated with 12 (5.43 g, 21 mmol, 0.74 equiv). After 5 min, tert-butyl (3-hydroxypropyl)carbamate (4.88 ml, 29 mmol) in DCM was added. The reaction was stirred for 1 h and quenched with 10% HCl. The reaction mixture was extracted with EtOAc and the organic layer was washed with NaHCO₃(sat). The organics were dried with NaCl(sat) and Na₂SO₄(s) and removed under reduced pressure. The residue was purified by column chromatography utilizing an ISCO system (EtOAc-Hexane) to give 4.54 g (76%); m/z 286.

### Method 27

### 2-Methyl-2-(2-thienyl)propanenitrile

A solution of NaH (0.974 g, 24.36 mmol) in DMSO (30 ml) was treated with 2-thienylacetonitrile (1.00 g, 8.12 mmol) by dropwise addition. After stirring for 5 min at 25° C., methyl iodide (6.91 g, 48.72 mmol) was added to the reaction mixture via syringe. The resulting solution was stirred at 25° C. for 3 h before being diluted with H₂O (100 ml). The resulting solution was extracted with EtOAc. The organics were washed with NaCl(sat) and dried with MgSO₄(s). The organics were removed under reduced pressure, and the residue was purified by column chromatography utilizing an ISCO system (EtOAc-Hexane) to give 1.0 g of (81%) a pale yellow oil; m/z 152.

### Method 28

### 2-(5-Formyl-2-thienyl)-2-methylpropanenitrile

A solution of 2-methyl-2-(2-thienyl)propanenitrile (Method 27; 0.260 g, 1.71 mmol) in THF (5.8 ml) was cooled to −78° C. and treated with tert-butyl lithium (1.7 M solution in pentanes; 1.26 ml, 2.14 mmol) by dropwise addition. The resulting bright yellow mixture was stirred for 1 h and treated with DMF (0.330 ml, 4.27 mmol) via syringe. The reaction mixture was stirred for 6 h at −78° C. and then quenched by the addition of NH₄Cl(sat) (25 ml). The resulting mixture was extracted with EtOAc and the organics were washed with NaCl(sat) and dried with MgSO₄(s). The organics were removed under reduced pressure to give 0.271 g of (88%) a colourless oil; m/z 180.

### Method 29

### 5-(1-Cyano-1-methylethyl)thiophene-2-carboxylic acid

A solution of 2-(5-formyl-2-thienyl)-2-methylpropanenitrile (Method 28; 0.271 g, 1.51 mmol) in tert-butyl alcohol (7.5 ml) and 2-methyl-2-butene (4.5 ml) was treated with. a solution of NaClO₂ (1.22 g, 13.60 mmol) and NaH₂PO₄ (1.45 g, 10.57 mmol) (7 ml). The reaction mixture was stirred for 30 min at 25° C. and then the organics were removed under reduced pressure. The residue was washed with NaHCO₃(sat) and extracted with EtOAc. The organics were washed with NaCl(sat) and dried with MgSO₄(s). The organics were removed under reduced pressure to give 0.265 g (90%) of a white solid; m/z 196.

### Method 30

### 2-Amino-4-methoxy benzoic acid

A mixture of 4-methoxy-2-nitrobenzoic acid (20 g, 101.5 mmol), 10% Pd/C (1.5 g) in MeOH (200 ml) was stirred at 25° C. under a hydrogen atmosphere for 168 h. The mixture was diluted with MeOH and filtered through diatomaceous earth. The organics were removed under reduced pressure to yield a light brown solid (14.85 g, 87.6%). NMR: 7.65 (d, 1H), 6.30 (s, 1H), 6.15 (d, 1H), 3.80 (s, 3H); m/z 167.

### Method 31

### 7-Methoxy-3H-quinazolin-4-one

A mixture of 2-amino-4-methoxy benzoic acid (Method 30; 4.85 g, 88.9 mmol) and formamidine acetate (18.49 g, 177.8 mmol) in 2-methoxyethanol (100 ml) was stirred at reflux for 12 h. The reaction mixture was cooled to 25° C. and diluted with 0.01 M ammonia (100 ml). The mixture was then stirred at 25° C. for 30 min and the resulting solid was collected by filtration. The solid was washed with 0.01M ammonia and water. The product was dried by high vacuum to obtain a light brown solid 11.5 g (73%). NMR: 12.10 (s, br, 1H), 8.05 (m, 2H), 7.10 (m, 2H), 3.90 (s, 3H); m/z 167.

### Method 32

### 4-Chloro-7-methoxy-quinazoline

7-Methoxy-3H-quinazolin-4-one (Method 31; 11.5 g, 65.3 mmol) was suspended in thionyl chloride (100 ml) and DMF (0.1 ml). The reaction mixture was heated to reflux for 3.5 h. The organics were removed under reduced pressure to give a light yellow solid (13.8 g); m/z 195.

### Methods 33-46

The following compounds were prepared by the procedure of Method 32 using the appropriate starting materials.

| Meth | Compound | M/z | S.M |
|---|---|---|---|
| 33 | 4-chloro-quinazoline | 164 | 2-Amino-benzoic acid |
| 34 | 4-chloro-6-methoxy-quinazoline | 194 | 2-Amino-5-methoxy-benzoic acid |
| 35 | 4-chloro-8-methoxy-quinazoline | 194 | 2-Amino-3-methoxy-benzoic acid |
| 36 | 4-chloro-5-methoxy-quinazoline | 194 | 2-Amino-6-methoxy-benzoic acid |
| 37 | 4-chloro-7-trifluoromethyl- | 232 | 2-Amino-4-trifluoromethyl-benzoic |
| | quinazoline | | acid |
| 38 | 4-chloro-7-fluoro-quinazoline | 182 | 2-Amino-4-fluoro-benzoic acid |
| 39 | 4-chloro-7-nitro-quinazoline | 211 | 2-Amino-4-nitro-benzoic acid |
| 40 | 4-chloro-7-bromo-quinazoline | 243 | 2-Amino-4-bromo-benzoic acid |
| 41 | 4-chloro-7-chloro-quinazoline | 199 | 2-Amino-4-chloro-benzoic acid |
| 42 | 4-chloro-7-methyl-quinazoline | 178 | 2-Amino-4-methyl-benzoic acid |
| 43 | 4-chloro-5,7-dimethoxy-quinazoline | 224 | 2-Amino-4,6-methoxy-benzoic acid |
| 44 | 4-chloro-5-fluoro-quinazoline | 182 | 2-Amino-6-fluoro-benzoic acid |
| 45 | 4-chloro-6-methyl-quinazoline | 178 | 2-Amino-5-methylbenzoic acid |
| 46 | 4-chloro-6-hydroxy-7-methoxy- | 210 | 2-Amino-4-methoxy-5-hydroxy |
| | quinazoline | | benzoic acid |

### Method 47

### 4-Dimethylaminomethyl-5-trifluoromethyl-benzoic acid methyl ester

A mixture of 4-bromomethyl-3-trifluoromethyl-benzoic acid methyl ester (Method 58; 252 mg, 0.85 mmol), dimethylamine (2.0 M in THF; 2 ml, 4 mmol) and K₂CO₃ (235 mg, 1.70 mmol) in MeCN (10 ml) was stirred at 80° C. for 4 h. The heterogeneous mixture was filtered and the solids were washed with MeOH. The organics were concentrated under reduced pressure and the residue was purified by column chromatography utilizing an ISCO system (hexane-EtOAc) to give 72 mg (41%) of a colourless oil. NMR: 8.25 (d, 1H), 8.20 (s, 1H), 7.95 (d, 1H), 3.90 (s, 3H), 3.60 (s, 2H), 2.18 (s, 6H); m/z 261.

### Method 48

### 4-Dimethylaminomethyl-3-trifluoromethyl-benzoic acid

A solution of 4-dimethylaminomethyl-5-trifluoromethyl-benzoic acid methyl ester (Method 47; 72 mg, 0.3 mmol) in THF-MeOH—H₂O (3:1:1; 5 ml) was treated with lithium hydroxide (22 mg, 0.919 mmol) in H₂O (2 ml). The reaction mixture was stirred at 25° C. for 12 h. The organics were removed under reduced pressure. NMR: 13.00 (s, br, 1H), 8.45 (d, 1H), 8.21 (m, 2H), 4.50 (s, 2H), 2.73 (s, 6H); m/z 247.

### Method 49

### 5-Fluoro-isophthalic acid

3-Fluoro-5-methyl-benzoic acid (2 g, 13 mmol) and KMnO₄ (8.22 g, 52 mmol) were dissolved in water (200 ml), and the reaction mixture was heated at reflux for 12 h. The hot reaction mixture was then filtered through diatomaceous earth. The resultant solution was cooled to 25° C. and then acidified with HCl (conc). The resulting solid was collected by vacuum filtration to give 2.4 g (100%). NMR: 8.25 (s, 1H), 7.88 (d, 2H).

### Method 50

### 5-Fluoroisophthalic acid dimethyl ester

A solution of 5-fluoroisophthalic acid (Method 49; 1.3 g, 7.1 mmol) in MeOH (30 ml) was treated with sulfuric acid (conc) (0.25 ml). The reaction mixture was then refluxed for 12 h. The organics were removed under reduced pressure and the residue was then neutralized with NaHCO₃(sat) and extracted with DCM. The organics were washed with NaCl(sat) and dried with Na₂SO₄(s) and then concentrated under reduced pressure to give 1.25 g (83%) as white solid. NMR: 8.42 (s, 1H), 7.88 (d, 2H), 3.90 (s, 6H).

### Method 51

### 3-Fluoro-5-hydroxymethyl-benzoic acid methyl ester

A solution of 5-fluoroisophthalic acid dimethyl ester (Method 50; 301 mg, 1.42 mmol) in THF (15 ml) at 0° C. was treated with lithium aluminium hydride (30 mg, 0.7 mmol). The reaction mixture stirred at 25° C. for 2 h. The reaction mixture was quenched with H₂O and extracted with EtOAc. The organics were washed with NaCl(sat) and dried with Na₂SO₄(s) and then concentrated under reduced pressure. The residue was purified by column chromatography utilizing an ISCO system (hexane-EtOAc) to give (120 mg, 50%) of a colourless oil. NMR: 780 (s, 1H), 7.62 (d, 1H), 7.31 (d, 1H), 4.76 (s, 2H), 3.95 (s, 3H), 1.85 (s, br, 1H).

### Method 52

### 3-Fluoro-5-methanesulfonyloxymethyl-benzoic acid methyl ester

A solution of 3-fluoro-5-hydroxymethyl-benzoic acid methyl ester (Method 51; 120 mg, 0.65 mmol) in DCM (5 ml) at 0° C. was treated with methanesulfonyl chloride (148 mg, 1.3 mmol) and triethylamine (198 mg, 1.96 mmol). The reaction mixture was stirred at 25° C. for 0.5 h. The organics were removed under reduced pressure and residue was purified by column chromatography utilizing an ISCO system (hexane-EtOAc) to give (166 mg, 97%) of a colourless oil. NMR: 7.79 (s, 1H), 7.17 (d, 1H), 7.26 (d, 1H), 5.19 (s, 2H), 3.87 (s, 3H), 2.95 (s, 3H).

### Method 53

### 3-Cyanomethyl-5-fluoro-benzoic acid methyl ester

A solution of 3-fluoro-5-methanesulfonyloxymethyl-benzoic acid methyl ester (Method 52; 50 mg, 0.19 mmol) in MeCN (2 ml) was treated with sodium cyanide (19 mg, 0.38 mmol) and 18-crown-6 (10 mg). The reaction mixture was stirred at 65° C. for 2 h. The heterogeneous mixture was filtered and the solids were washed with DCM. The organics were concentrated under reduced pressure and the residue was purified by column chromatography utilizing an ISCO system (hexane-EtOAc) to give (30 mg, 81.7%) of a colourless oil. NMR: 7.78 (s, 1H), 7.65 (d, 1H), 7.20 (d, 1H), 3.90 (s, 3H), 3.78 (s, 2H).

### Method 54

### 3-(Cyano-dimethyl-methyl)-5-fluoro-benzoic acid methyl ester

A solution of 3-cyanomethyl-5-fluoro-benzoic acid methyl ester (Method 53; 1.7 g, 8.79 mmol) in DMSO (50 ml) under nitrogen was treated with sodium hydride (60% dispersed in mineral oil; 1.05 g, 26.4 mmol). The reaction mixture was cooled to 0° C. and methyl iodide (12.49 g, 5.49 ml, 87.9 mmol) was added dropwise. The reaction mixture was stirred at 25° C. for 5 h and then quenched with H₂O. The reaction mixture was then extracted with EtOAc and the organics were washed with NaCl(sat), dried with Na₂SO₄(s) and then concentrated under reduced pressure. The residue was purified by column chromatography utilizing an ISCO system (hexane-EtOAc) to give 1.94 g (100%) of a colourless oil. NMR: 7.82 (s, 1H), 7.58 (d, 1H), 7.31 (d, 1H), 3.89 (s, 3H), 1.70 (s, 6H).

### Method 55

### 3-(Cyano-dimethyl-methyl)-5-fluoro-benzoic acid

A solution of 3-(cyano-dimethyl-methyl)-5-fluoro-benzoic acid methyl ester (Method 54; 1.94 g, 8.79 mmol) in THF-MeOH—H₂O (3:1:1; 50 ml) was treated with lithium hydroxide (633 mg, 26.4 mmol) in H₂O (5 ml). The reaction mixture was stirred at 25° C. for 12 h. The organics were removed under reduced pressure and then H₂O was added. The reaction was then acidified with 10% HCl and the resulting solid was collected by vacuum filtration to give 1.8 g (100%) as a white solid. NMR: 7.95 (s, 1H), 7.68 (d, 1H), 7.41 (d, 1H), 1.70 (s, 6H).

### Method 56

### 3-Cyclopropyl-5-fluorobenzoic acid

3-Bromo-5-Fluorobenzoic acid (1.00 g, 4.57 mmol), cyclopropylboronic acid (1.18 g, 13.71 mmol, 3.0 equiv) and K₃PO₄(7.76 g, 36.56 mmol, 8.0 equiv) in toluene-H₂O (25:1, 31 ml) were treated with Pd(Ph₃P)₄ (1.05 g, 0.912 mmol, 20 mol %). The reaction mixture was heated to 100° C. for 12 h. The reaction was quenched with 10% NaOH and extracted with EtOAc. The aqueous layer was acidified with 10% HCl and extracted with EtOAc. The organics were dried with NaCl(sat) and Na₂SO₄(s) and removed under reduced pressure; m/z 181.

### Method 57

### 4-Methyl-3-trifluoromethyl-benzoic acid methyl ester

A slurry of potassium hydroxide (84 mg, 1.5 mmol) in DMSO was treated with a solution of 4-methyl-3-trifluoromethyl-benzoic acid (306 mg, 1.5 mmol) in DMSO (5 ml). The resulting mixture was stirred for 15 min and cooled with an ice bath. After the addition of methyl iodide (426 mg, 3 mmol), the mixture was stirred for 2 h at 25° C. The reaction mixture was quenched with water and extracted with EtOAc. The organics were washed with NaCl(sat), dried with Na₂SO₄(s) and concentrated under reduced pressure to give 327 mg (100%). NMR: 8.10 (m, 2H), 7.60 (s, 1H), 3.86 (s, 3H), 2.45 (s, 3H); m/z 218

### Method 58

### 4-Bromomethyl-3-trifluoromethyl-benzoic acid methyl ester

A suspension of 4-methyl-3-trifluoromethyl-benzoic acid methyl ester (Method 57; 327 mg, 1.5 mmol), N-bromosuccinimide (267 mg, 1.5 mmol) and benzoyl peroxide (catalytic) in CCl₄ (10 ml) was heated to reflux for 3 h. The reaction mixture was cooled to 25° C. and filtered through a pad of silica gel. The organics were removed under reduced pressure and the residue was purified by column chromatography utilizing an ISCO system (hexane-EtOAc) to give 252 mg (56.5%) of a colourless oil. NMR: 7.70-8.25 (m, 3H), 4.85 (s, 2H), 3.91 (s, 3H); m/z 297.

### Method 59

### 2-Methyl-2-(4-methylpyridin-2-yl)propanenitrile

A solution of 2-fluoro-4-methylpyridine (1.00 g, 9.00 mmol), 2-methylpropanenitrile (2.48 g, 36 mmol) in toluene (30 ml) was treated with potassium hexamethyldisilazide (13.5 mmol) and the reaction was refluxed for 1 h. The reaction was quenched with NH₄Cl(sat) and extracted with EtOAc. The organics were dried with MgSO₄(s) and concentrated under reduced pressure. The residue was purified by column chromatography utilizing an ISCO system (hexane-EtOAc) to give 0.870 g (60%) of a colourless oil; m/z 161.

### Method 60

### 2-(1-Cyano-1-methylethyl)isonicotinic acid

A solution of 2-methyl-2-(4-methylpyridin-2-yl)propanenitrile (Method 59; 0.870 g, 5.43 mmol) in H₂O (15 ml) at 60° C. was treated with KMnO₄ (4.3 g, 27 mmol). The reaction was heated to reflux for 2 h and then filtered through diatomaceous earth. The pH was adjusted to 4 by addition of 1N HCl and the aqueous phase was extracted with EtOAc. The organics were dried with MgSO₄(s) and concentrated under reduced pressure. The residue was purified by column chromatography utilizing an Isco system (EtOAc-MeOH) to give 0.700 g (68%) of a white solid: m/z 191.

### Method 61

The following compounds were prepared by the procedure of Method 60, using the appropriate starting material.

| Meth | Compound | m/z | SM |
|---|---|---|---|
| 61 | 3-(1-Cyano-1-methylethyl)-2-fluorobenzoic acid | 208 | Method |
| | | | 15 |

### Method 62

### N³-(7-Methoxy-quinazolin-4-yl)-4-methyl-benzene-1,3-diamine

A suspension of (7-methoxy-quinazolin-4-yl)-(2-methyl-5-nitro-phenyl)-amine (Method 63; 4.6 g, 14.8 mmol) and 10% Pd/C (500 mg) in MeOH (200 ml) was stirred at 25° C. under hydrogen for 12 h. The reaction mixture filtered through a diatomaceous earth and concentrated under reduced pressure to 5 ml. EtOAc (5 ml) was added to the solution and the resulting solid was collected by vacuum filtration to give 2.5 g (60.2%) of a yellow solid; m/z 280.

### Method 63

### N³-(7-Methoxy-quinazolin-4-yl)-(2-methyl-5-nitro-phenyl-amine

A mixture of 4-chloro-7-methoxy-quinazoline (Method 32; 3.5 g, 18 mmol) and 2-methyl-5-nitro-phenylamine (2.3 g, 15 mmol) in isopropanol (150 ml) was refluxed for 12 h. The reaction mixture was cooled to 25° C. and the resulting precipitate was collected by vacuum filtration. The solid was washed with ether and dried under reduced pressure to give 4.6 g (98.9%) of a light yellow solid. NMR: 11.55 (s, br, 1H), 8.85 (s, 1H), 8.75 (d, 1H), 8.30 (s, 1H), 8.20 (d, 1H), 7.75 (d, 1H), 7.52 (d, 1H), 7.30 (s, 1H), 4.02 (s, 3H), 2.40 (s, 3H); m/z 310.

### Method 64

### Ethyl 3-formyl-4-oxobutanoate

A solution of ethyl formate (10.0 g, 367.9 mmol) in anhydrous diethyl ether was treated sodium hydride (60% in mineral oil) (1.8 g, 44.2 mmol). The reaction mixture was cooled to 0° C. and ethyl 3-ethoxy-3-methoxypropanoate (7.0 g, 36.8 mmol) was added. The reaction mixture was stirred at 0° C. for 5 h and then at 25° C. for 12 h. The reaction mixture was quenched with cold H₂O and extracted with diethyl ether. The aqueous layer was then acidified with 10% HCl and then extracted with DCM. The organic layer was dried over Na₂SO₄ and concentrated under reduced pressure. The crude product (3.3 g, 57%) was used directly. ¹H NMR (300 MHz): 1.29 (t, 3H), 4.24 (q, 2H), 9.08 (s, 2H).

### Method 65

### Ethyl 1-tert-butyl-1H-pyrazole-3-carboxylate

A solution of ethyl 3-formyl-4-oxobutanoate (Method 64; 350 mg, 2.2 mmol) in EtOH (5 ml) was treated with triethylamine (465 μL, 3.3 mmol) and t-butyl hydrazine hydrochloride. The reaction stirred for 12 h at 25° C. EtOH was removed under reduced pressure and the residue was redissolved in EtOAc and washed with H₂O. The organics were dried with Na₂SO₄(s) and concentrated under reduced pressure. The residue was purified by column chromatography utilizing an Isco system (5% MeOH in CH₂Cl₂) to yield 327 mg (76%) of an oil. ¹H NMR (300 MHz): 1.29-1.35 (m, 3H), 1.57 (s, 9H), 4.25 (q, 2H), 7.86 (s, 1H), 8.20 (s, 1H).

### Method 66

### 1-tert-Butyl-1H-pyrazole-4-carboxylic acid

A solution of ethyl 1-tert-butyl-1H-pyrazole-3-carboxylate (Method 65; 327 mg, 1.66 mmol) in THF-MeOH—H₂O (3:1:1, 8 ml) was treated with LiOH (120 mg, 5.0 mmol). The reaction mixture was stirred at 25° C. for 12 h. H₂O and EtOAc were added to the reaction mixture and the resulting solution was acidified with 10% HCl. The organics were dried with Na₂SO₄(s) and concentrated under reduced pressure to yield 217 mg (78%). m/z 168.

### Method 67

### 5,7-Dimethoxy-3H-quinazolin-4-one

A suspension of 5,7-difluoro-3H-quinazolin-4-one (1 g, 5.49 mmol) in anhydrous DMF (15 ml) was treated with sodium methoxide (890 mg, 16.47 mmol, 3 equiv). The reaction mixture was stirred at 25° C. for 30 min and then at 90° C. for 5 h. The reaction mixture was poured into 10% ammonium chloride (100 ml) and the resulting precipitate was collected by vacuum filtration to yield a white solid (1.13 g, 100%). NMR (400 MHz, DMSO-d₆): 11.70 (s, br, 1H), 7.90 (s, 1H), 6.62 (s, 1H), 6.50 (s, 1H), 3.88 (s, 3H), 3.80 (s, 3H); m/z: 206.

### Method 68

### 3-Fluoro-5-isopropylbenzoic acid

3-Cyclopropyl-5-fluorobenzoic acid (450 mg, 2.50 mmol) and PtO₂ (20 mg) in AcOH (10 ml) were shaken under 45 psi hydrogen in a Parr hydrogenator for 3 h. The reaction mixture was filtered through diatomaceous earth, and the resulting filtrate was concentrated under reduced pressure giving the desired compound (400 mg, 88%); m/z 181.

### Method 69

### N-(3-Amino-4-methylphenyl)-3-(trifluoromethyl)benzamide hydrochloride

N-(4-Methyl-3-nitrophenyl)-3-(trifluoromethyl)benzamide (Method 70) (3.7 g, 11.41 mmol) and 10% palladium on carbon (370 mg) in methanol (20 ml) was shaken under 40 psi H₂ for 3 hours. The reaction mixture was then filtered over diatomaceous earth and the solvent was removed under reduced pressure. The residue was taken up in 30 ml 4 N HCl in dioxane and the solvent was removed under reduced pressure to afford the title compound (3.66 g, 97%). m/z 295.

### Method 70

### N-(4-Methyl-3-nitrophenyl)-3-(trifluoromethyl)benzamide

3-(Trifluoromethyl)benzoyl chloride (2.70 g, 12.95 mmol) in 10 ml anhydrous DCM was added to 4-methyl-3-nitroaniline (1.9 g, 12.95 mmol), and TEA (5.4 ml, 38.85 mmol) in DCM (65 ml) and the reaction mixture was allowed to stir at 25° C. for 1 h. The resulting mixture was washed with 1 N HCl, water and brine. The organic extracts were dried and solvent was removed under reduced pressure to give the title compound as a pale yellow solid (3.70 g, 88%). m/z 325.

1. A compound of formula (I): 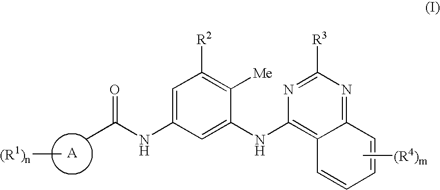
wherein:
Ring A is phenyl or a 5- or 6-membered heteroaryl; wherein if said heteroaryl contains an —NH— moiety that nitrogen may be optionally substituted by a group selected from R⁵;
R¹ is a substituent on carbon and is selected from halo, nitro, hydroxy, trifluoromethoxy, amino, carboxy, carbamoyl, mercapto, sulphamoyl, C₁₋₆alkyl, C₂₋₆alkenyl,
C₂₋₆alkynyl, C₁₋₆alkoxy, C₁₋₆alkanoyl, C₁₋₆alkanoyloxy, N-(C₁₋₆alkyl)amino,
C₁₋₆alkanoylamino, N-(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl or carbon linked heterocyclyl; wherein R¹ may be optionally substituted on carbon by one or more R⁸; and wherein if said heterocyclyl contains an —NH— moiety that nitrogen may be optionally substituted by a group selected from R⁹;
n is selected from 1-4; wherein the values of R¹ may be the same or different;
R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N-(C₁₋₆alkyl)₂-carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁰— or heterocyclyl-R¹¹—; wherein R² may be optionally substituted on carbon by one or more R¹²; and wherein if said heterocyclyl contains an —NH— moiety that nitrogen may be optionally substituted by a group selected from R¹³;
R³ and R⁴ are substituents on carbon and are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N-(C₁₋₆alkyl)₂-carbamoyl, C₁₋₆alkylS(O)₃ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁴— or heterocyclyl-R¹⁵—; wherein R⁴ may be optionally substituted on carbon by one or more R¹⁶; and wherein if said heterocyclyl contains an —NH— moiety that nitrogen may be optionally substituted by a group selected from R¹⁷;
m is selected from 0-4; wherein the values of R⁴ may be the same or different;
R⁸ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N-(C₁₋₆alkyl)₂-carbamoyl, C₁₋₆alkylS(O)₃ wherein a is 0 to 2, C₁₋₆alkoxycarbonyl, N-(C₁₋₆alkyl)sulphamoyl, N,N—(C₁₋₆alkyl)₂sulphamoyl, C₁₋₆alkylsulphonylamino, carbocyclyl-R¹⁸— or heterocyclyl-R¹⁹—; wherein R⁸ and R¹² independently of each other may be optionally substituted on carbon by one or more R²⁰; and wherein if said heterocyclyl contains an —NH— moiety that nitrogen may be optionally substituted by a group selected from R²¹;
R¹⁶ is selected from halo, nitro, cyano, hydroxy, trifluoromethoxy, amino, carboxy, carbamoyl, mercapto, sulphamoyl, C₁₋₁₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂-amino, C₁₋₆alkanoylamino, N-(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R²²— or heterocyclyl-R²³—;
wherein R¹⁶ may be optionally substituted on carbon by one or more R²⁴; and wherein if said heterocyclyl contains an —NH— moiety that nitrogen may be optionally substituted by a group selected from R²⁵;
R¹⁰, R¹¹, R¹⁴, R¹⁵, R¹⁸, R¹⁹, R²² and R²³ are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ are independently selected from hydrogen or C₁₋₆alkyl and s is 0-2;
R⁵, R⁹, R¹³, R¹⁷, R²¹ and R²⁵ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N-(C₁₋₆alkyl)carbamoyl, benzyl, benzyloxycarbonyl, benzoyl and phenylsulphonyl;
R²⁰ and R²⁴ are independently selected from halo, nitro, cyano, hydroxy, trifluoromethoxy, trifluoromethyl, amino, carboxy, carbamoyl, mercapto, sulphamoyl, methyl, ethyl, hydroxymethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; or a pharmaceutically acceptable salt thereof,
with the proviso that said compound is not N-{3-[(6,7-dimethoxyquinazolin-4-yl)amino]-4-methylphenyl}-3-(trifluoromethyl)benzamide.

2. A compound of formula (I) or a pharmaceutically acceptable salt thereof, as claimed in claim 1, wherein Ring A is phenyl or a 5- or 6-membered heteroaryl; wherein if said heteroaryl contains an —NH— moiety that nitrogen may be optionally substituted by a group selected from R⁵; wherein R⁵ is C₁₋₆alkyl.

3. A compound of formula (I) or a pharmaceutically acceptable salt thereof, as claimed in claim 1, wherein R¹ is a substituent on carbon and is selected from halo, C₁₋₆alkyl, C₁₋₆alkylS(O)ₐ wherein a is 2, N,N-(C₁₋₆alkyl)₂sulphamoyl, carbocyclyl or carbon linked heterocyclyl; wherein R¹ may be optionally substituted on carbon by one or more R⁸; wherein R⁸ is selected from halo, cyano or N,N-(C₁₋₆alkyl)₂-amino.

4. A compound of formula (I) or a pharmaceutically acceptable salt thereof, as claimed in claim 1, wherein n is selected from 1 or 2; wherein the values of R¹ may be the same or different.

5. A compound of formula (I) or a pharmaceutically acceptable salt thereof, as claimed in any one of claim 1, wherein R³ and R⁴ are substituents on carbon and are independently selected from hydrogen, halo, nitro, hydroxy, amino, carboxy, C₁₋₆alkyl and C₁₋₆alkoxy; wherein R⁴ may be optionally substituted on carbon by one or more R¹⁶; wherein
R¹⁶ is selected from halo, amino, C₁₋₆alkoxy, N,N-(C₁₋₆alkyl)₂amino, C₁₋₆alkoxycarbonylamino, carbocyclyl-R²²— or heterocyclyl-R²³—; wherein R¹⁶ may be optionally substituted on carbon by one or more R²⁴; and wherein if said heterocyclyl contains an —NH— moiety that nitrogen may be optionally substituted by a group selected from R²⁵;
R²² and R²³ are independently selected from a direct bond and —O—;
R²⁵ is selected from C₁₋₆alkyl and C₁₋₆alkoxycarbonyl;
R²⁴ is hydroxymethyl.

6. A compound of formula (I) or a pharmaceutically acceptable salt thereof, as claimed in claim 1, wherein m is selected from 0-2; wherein the values of R⁴ may be the same or different.

7. A compound of formula (I): 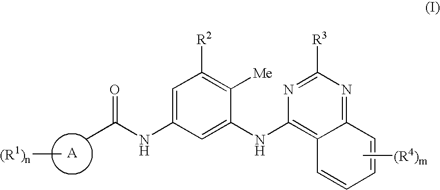
wherein:
Ring A is phenyl, thien-2-yl, 1-t-butyl-1H-pyrazol-4-yl, 1-t-butyl-1H-pyrazol-5-yl or pyrid-4-yl;
R¹ is a substituent on carbon and is selected from fluoro, chloro, methyl, trifluoromethyl, 1-methyl-1-cyanoethyl, 1-cyanocyclobutyl, 4-cyano-2,3,5,6-tetrahydropyran-4-yl, 1-cyanocyclopropyl, isopropyl, mesyl, N,N-dimethylsulphamoyl, dimethylaminomethyl and cyclopropyl;
n is selected from 1 or 2; wherein the values of R¹ may be the same or different;
R² is hydrogen;
R³ and R⁴ are substituents on carbon and are independently selected from hydrogen, fluoro, chloro, bromo, nitro, hydroxy, amino, carboxy, methyl, methoxy, benzyloxy, 3-aminopropoxy, 3-morpholinopropoxy, 2-methoxyethoxy, 1-methylpyrrolidin-2-ylmethoxy, piperidin-4-ylmethoxy, piperidin-3-ylmethoxy, azetidin-2-ylmethoxy, 1-t-butoxycarbonylazetidin-2-ylmethoxy, azetidin-3-ylmethoxy, 1-t-butoxycarbonylazetidin-3-ylmethoxy, pyrrolidin-2-ylmethoxy, 1-t-butoxycarbonylpyrrolidin-2-ylmethoxy, pyrrolidin-3-yloxy, 1-t-butoxycarbonylpyrrolidin-3-yloxy, 2-(2-hydroxymethylpyrrolidin-1-yl)ethoxy, 3-(2-hydroxymethylpyrrolidin-1-yl)propoxy, 3-dimethylaminopropoxy, trifluoromethyl, propoxy, isopropoxy, 3-(t-butoxycarbonylamino)propoxy, 3-bromopropoxy, 1-(t-butoxycarbonyl)piperidin-4-ylmethoxy and 1-(t-butoxycarbonyl)piperidin-3-ylmethoxy;
m is selected from 0-2; wherein the values of R⁴ may be the same or different;
or a pharmaceutically acceptable salt thereof,
with the proviso that said compound is not N-{3-[(6,7-dimethoxyquinazolin-4-yl)amino]-4-methylphenyl}-3-(trifluoromethyl)benzamide.

8. A compound of formula (I): 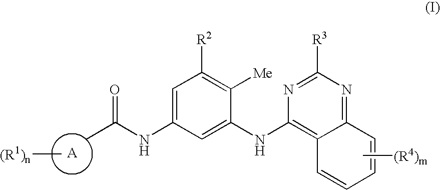
selected from:
3-(cyano-dimethyl-methyl)-N-[3-(7-methoxy-quinazolin-4-ylamino)-4-methyl-phenyl]-benzamide;
3-(cyano-dimethyl-methyl)-5-fluoro-N-[3-(7-methoxy-quinazolin-4-ylamino)-4-methyl-phenyl]-benzamide;
3-(1-cyano-1-methylethyl)-2-fluoro-N-{3-[(7-methoxy quinazolin-4-yl)amino]-4-methylphenyl}benzamide;
3-(cyano-dimethyl-methyl)-N-[3-(5,7-dimethoxy-quinazolin-4-ylamino)-4-methyl-phenyl]-benzamide;
3-(1-cyano-1-methylethyl)-N-{3-[(7-isopropoxyquinazolin-4-yl)amino]-4-methyl phenyl}benzamide;
N-{3-[6,7-dimethoxyquinazolin-4-ylamino]-4-methylphenyl}-3-fluoro-5-isopropylbenzamide;
2-(cyano-dimethyl-methyl)-N-[3-(7-methoxy-quinazolin-4-ylamino)-4-methyl-phenyl]-isonicotinamide;
3-(cyano-dimethyl-methyl)-N-{3-[7-(3-dimethylamino-propoxy)-quinazolin-4-ylamino]-4-methyl-phenyl}-benzamide;
4-dimethylaminomethyl-N-[3-(7-methoxy-quinazolin-4-ylamino)-4-methyl-phenyl]-3-trifluoromethyl-benzamide; and
3-(cyano-dimethyl-methyl)-N-[3-(7-methyl-quinazolin-4-ylamino)-4-methyl-phenyl]-benzamide;
or a pharmaceutically acceptable salt thereof.

9. A process for preparing a compound of formula (I) or a pharmaceutically acceptable salt thereof as claimed in claim 1, which process, wherein the variables are, unless otherwise specified, as defined in claim 1, comprises of:
Process a) reacting an amine of the formula (II) 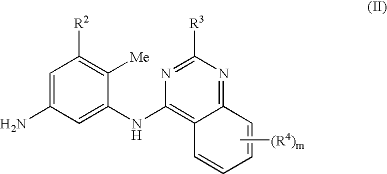
with an acid of formula (III): 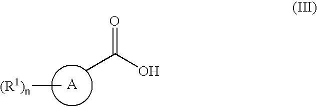
or an activated acid derivative thereof,
Process b) reacting an amine of formula (IV): 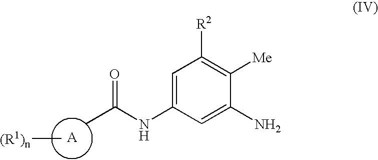
with a compound of formula (V): 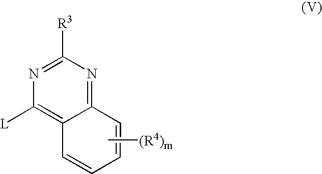
wherein L is a displaceable group
Process c) reacting an amine of formula (VI): 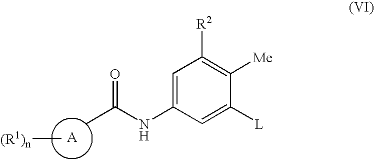
with a compound of formula (VII): 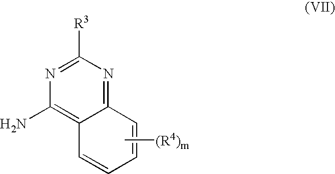
and thereafter if necessary:
i) converting a compound of the formula (I) into another compound of the formula (I);
ii) removing any protecting groups;
iii) forming a pharmaceutically acceptable salt.

10. A pharmaceutical composition which comprises a compound of the formula (I), or a pharmaceutically acceptable salt thereof, as claimed in claim 1, in association with a pharmaceutically-acceptable diluent or carrier.

11. (canceled)

12. (canceled)

13. (canceled)

14. (canceled)

15. A method for producing a B-Raf inhibitory effect in a warm-blooded animal, such as man, in need of such treatment which comprises administering to said animal an effective amount of a compound of formula (I), or a pharmaceutically acceptable salt thereof, as claimed in claim 1.

16. A method for producing an anti-cancer effect in a warm-blooded animal, such as man, in need of such treatment which comprises administering to said animal an effective amount of a compound of formula (I), or a pharmaceutically acceptable salt thereof, as claimed in claim 1.

17. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof, as claimed in claim 1.

18. A pharmaceutical composition which comprises a compound of the formula (I), or a pharmaceutically acceptable salt thereof, as claimed in claim 1, in association with a pharmaceutically-acceptable diluent or carrier for use in the production of a B-Raf inhibitory effect in a warm-blooded animal such as man.

19. A pharmaceutical composition which comprises a compound of the formula (I), or a pharmaceutically acceptable salt thereof, as claimed in claim 1, in association with a pharmaceutically-acceptable diluent or carrier for use in the production of an anti-cancer effect in a warm-blooded animal such as man.

20. A pharmaceutical composition which comprises a compound of the formula (I), or a pharmaceutically acceptable salt thereof, as claimed in claim 1, in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.
